SEQUENCE LISTING (3 sequences)

SEQ ID NO: 1 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 2 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 3 - protein, 501 aa, Homo sapiens
MRLPKLLTFLLWYLAWLDLEFICTVLGAPDLGQRPQGSRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAVPRSRRAAQLKLSSCPSGRQPAALLDVRSVPGLDGSGWEVFDIWKLFRNFKNSAQLCLELEAWERGRTVDLRGLGFDRAARQVHEKALFLVFGRTKKRDLFFNEIKARSGQDDKTVYEYLFSQRRKRRAPLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

(19) United States
(12) Patent Application Publication
(10) Pub. No.: US 2010/0112028 A1
(43) Pub. Date: May 6, 2010

(54) Composite material for use as protein carrier

(76) Inventors: Klaus Hellerbrand, Geltendorf (DE); Michael Siedler, Munchen (DE); Andreas Schütz, Krailling (DE)

Correspondence Address: WOLF GREENFIELD & SACKS, P.C., 600 ATLANTIC AVENUE, BOSTON, MA, 02210-2206, US

(21) Appl. No.: 11/628,993

(22) PCT Filed: Jun. 9, 2005
(86) PCT No.: PCT/EP05/06206

§ 371 Date: Apr. 2, 2009

(30) Foreign Application Priority Data
Jun. 9, 2004 (EP) 04013667.3
Jun. 29, 2004 (EP) 04013669.9

(51) Int. Cl. A61K 9/00; A61K 9/16; A61K 47/02; A61K 38/02; B05D 3/00; A61P 19/00

(52) U.S. Cl. 424/423; 424/501; 424/400; 514/769; 514/2; 427/2.1; 427/2.26

(57) ABSTRACT

The present invention relates to a material having osteoinductive and osteoconductive properties in vivo comprising a ceramic carrier, preferably containing calcium phosphate, and an active agent, preferably an osteoinductive protein/peptide or a drug, and a polymer, wherein the active agent is homogeneously coated on the carrier and within the polymer, which is preferably a degradable polymer. Said polymer modulates the release kinetic of the active agent and protects same from degradation to prolong the half-life in vivo. Moreover, the present invention relates to a method for the production of a material having osteoinductive and osteoconductive properties in vivo.

The present invention relates to a material having osteoinductive and osteoconductive properties in vivo comprising a ceramic carrier, preferably containing calcium phosphate, and an active agent, preferably an osteoinductive protein/peptide or a drug, and a polymer, wherein the active agent is homogeneously coated on the carrier and/or within the polymer, which is preferably a degradable polymer.

Said polymer modulates the release kinetic of the active agent and protects same from degradation to prolong the half-life in vivo. Moreover, the present invention relates to a method for the production of a material having osteoinductive and osteoconductive properties in vivo. The invention encompasses a pharmaceutical composition comprising the material of the invention or a material which is obtainable by the method of the invention and relates to the use of said material for the preparation of a pharmaceutical composition for tissue regeneration, especially bone augmentation or treatment of bone defects, for treating degenerative and traumatic disc disease and for treatment of bone dehiscence. Finally, the invention relates to a kit comprising the material of the invention or a material, which is obtainable by the method of the invention.

### BACKGROUND TECHNOLOGY

In the field of medicinal technology many different materials have already been evaluated and/or are still in the process of being further improved for use as a bone replacement material (bone graft) by orthopaedic and maxillofacial surgeons. Because of the wide range of requirements the range of adequate materials is limited. Dependent on the indication and defect site in general an ideal bone graft substitute should have the following properties: the material must be biocompatible to promote cell adhesion and proliferation, preferably biodegradable and bioresorbable to be replaced completely by function tissue over time. Ideally the mechanical stability over time is similar to endogenous bone for bridging bone defects, filling cavities or bone augmentation, therefore the material has to be shapeable or mouldable to adapt the material to defect site. It should provide interconnected porosity to allow cell ingrowth to allow a binding to the surrounding bone tissue (osteoconductivity). Furthermore the material should be capable to act as a carrier for bone growth factors (BMPs) to allow the controlled release of these proteins to induce bone formation (osteoinductivity). Ideally the protein within or on the material is protected against washout and proteolytic degradation at the implantation site. Furthermore the material should be of synthetic origin to avoid infections and immunological reactions, should be available in access and of reliable quality. Finally the material should be clearly visible on radiographic examinations to survey the healing process and determine the amount and mass of new bone formation.

Nevertheless up to now there exists no material which is capable to fulfill all of these requirements of a preferable material and therefore the predominant treatment for bone defects is (still) the transposition of autologous bone (golden standard) from reservoirs of the patient's own body. Due to the medical need for artificial bone grafts and the limited availability of autologous bone, different materials are commonly in use despite their respective disadvantages. The most prominent are calcium phosphates and bioresorbable polymers of the poly(alpha-hydroxy esters) (e.g., PLGA). One has to keep in mind that the transplantation of autologous bone is regarded as a “golden standard” not because of ideal properties, but rather as a consequence of lacking alternatives.

### Calcium Phosphates Based Material

Various calcium phosphates ceramics such as beta-tricalcium phosphate (Ca₃(PO₄)₂) (beta-TCP), alpha-tricalcium phosphate (alpha-TCP), hydroxyapatite (Ca₁₀(PO₄)₆(OH)₂) (HA) and hydroxyapatite/β-TCP biphasic calcium phosphate (BCP) ceramics have been shown to be effective as bone replacement materials, because these ceramics are similar to the mineral phase of bone, in particular low crystalline hydroxyapatite. Manufacturing of calcium phosphate ceramics usually requires processing at high temperatures. The high temperatures are necessary to achieve special mineral phases of calcium phosphate (e.g., alpha-TCP or beta-TCP) and/or larger structures by sintering (e.g., blocks), or pyrolytic elimination of biological impurities (e.g., calcinated bovine bone). Most calcium phosphate ceramics are available only as granules or prefabricated blocks.

The bone replacement materials containing calcium phosphate are usually used when the regeneration of the bone is not possible any more (e.g., critical size defects) or is possible with difficulties only. In addition, bone replacement materials are used when the formation of additional bone is a prerequisite for a subsequent setting of an implant. Generally porous calcium phosphates exhibit an osteoconductive effect, because they represent a structure facilitating the migration of cells from the neighbouring bone. The presence of bones or different mesenchymal cells, however, is a precondition for the new formation of bones. Unfortunately calcium phosphates are brittle, have low tensile strength, low resistance to impact loading, and tend to fail when subject to repeated loading. Thus, although the chemical composition of calcium phosphates makes it very biocompatible, its mechanical properties make them less suited to serve in load-bearing applications. Unfortunately, granules can easily migrate into the surrounding tissue, and prefabricated materials are difficult to shape and may result in incomplete filling of the bone defect.

The effect of calcium phosphates can be significantly increased by adding autologous bone chips. These bones chips are not only osteoconductive, but also osteoinductive, i.e. they cause the transformation of undifferentiated mesenchymal stem cells into osteoblasts and chondrocytes. For reasons of safety, autogenic bone chips are preferred to the allogenic or xenogenic preparations. The use of autogenic bones, however, always involves a second surgical procedure, which is uncomfortable for the patient and is limited in access. In addition, biopsies of autologous bonegraft material have several disadvantages including post surgery pain and graft harvest complications.

Despite the above mentioned solid blocks and granules calcium phosphate cements (CPC) represent a further class of calcium phosphate based materials. They are delivered via injection or scraper into the bone defect as a moldable paste, can be adapted to the defect site and become solid after a period of time (Driessens et al., 2002). While CPC appears to have several advantages over presently used calcium phosphate biomaterials, an apparent limitation is its relatively long hardening time coupled with the washout effect explained below (Cherng et al., 1997). Another major problem of CPC is that they exhibit only micropores with pore sizes of submicrometer to a few micrometers. Previous studies with hydroxyapatite implants have shown that a pore size of about 100 μm to several hundred μm (macropores) are required for bone ingrowth (del Real et al., 2002). Macropores have been shown to be beneficial in facilitating cell infiltration and tissue ingrowth. However, macroporosity always results into a significant decrease in mechanical strength (Chow, 2000). It is generally accepted that CPC need further improvement to broaden their potential clinical applications. However, further improvement on material properties should keep in mind the way surgeons apply bone cements through minimal invasive surgery techniques (MIST) (Bohner, 2001).

In general CPC suffers from a relatively low mechanical stability (e.g. compression strength, brittleness) and lack of macroporosity e.g., osteoconductivity. The different cement reactions cause hydroxyapatite to form varying states of crystallinity which result in altered resorption time. Due to the lack of macroporosity and therefore osteoconductivity many of the cement formulations are poor carriers for osteogenic growth factors.

Further attempts have been made to improve CPC by adding biodegradable polymer (e.g. poly(DL-lactid-co-glycolic acid, PLGA) microparticles as delivery vehicles for bioactive molecules (Ruhe et al., 2003). Protein PLGA microparticles were added to the CPC powder and an aqueous solution of Na₂HPO₄ was used as a liquid, which was added to the composition shortly before application.

### Synthetic PLGA Based Material

Another important biomaterial class, which plays a predominant role in the field of bone tissue engineering are bioresorbable polymers (Vert, 1989; U.S. Pat. No. 6,214,021; U.S. Pat. No. 6,436,426). Especially the compound class of poly(hydroxy acids) has interesting application prospects due to their intrinsic biodegradability. These materials of which poly(glycolic acid) (PGA) and poly(lactic acid) (PLA) are the most prominent undergo hydrolytic chain cleavage (degradation) in a moist environment. Sustained degradation finally leads to the corresponding hydroxy acid units. Most of these hydrolytic end products occur as metabolites of many bacteria and cell phenotypes.

The degradation potential and their mechanical properties offer applications for the use as substrates for temporary implants in medical technology. Studies for various polymers in different tissues document the biocompatibility of these compounds in vivo forming the bases for the development of commercial implants as medical devices (Middleton et al., 2000).

In clinical surgery, polyesters presumably play the most important role in connection with the fixation, augmentation and replacement of bone. Devices like screws, plates, anchors or pins serve for positioning and fixation of bone fragments after bone loss or damage. The major feature of these absorbable polymers in application is the lacking necessity for a removal operation. Another important point is in favour of polymeric fixation devices: the mechanical integration of the implant in the bone tissue, but they are too flexible and too weak to meet the mechanical demands in many weight-bearing applications (Durucan et al., 2000).

An important drawback in totally polyester based implants is the possible accumulation of degradation products reaching cytotoxic levels and the accompanying acidification at the implant site due to the pH lowering release of acid monomers, especially when solid none porous implants were used and the degradation precedes according to a bulk degradation mechanism (Li et al., 1990).

To avoid such negative consequences caused by local pH decrease the implant should exhibit porosity e.g., by salt leaching process to avoid bulk degradation and therefore an accumulation of acidic monomers and to reduce the net amount of the polymer. Furthermore it has been suggested to incorporate basic salts within PLA/PGA implants (Agrawal et al., 1997).

For the use of these polymers as bone substitutes the common strategy is to design an implant which temporarily fulfils the function to allow a healing process and to retain strength during the early stages at the implantation site after operation. Afterwards the loss of strength and modulus of the implant should be in harmony with the increasing strength of the injured tissue (Tormälä et al., 1995). Proceeding degradation creates space for restoring processes to fill the gap with ingrown of vital host tissue. Presently, no filling material is available that fits this requirements satisfactorily to form new homogeneous bone in large defects (Rueger et al., 1996).

### Composite Materials

Because of the different strength and limitations of both materials there is a growing interest to combine the advantages of both, leading to the development of organic-inorganic systems, such as collagen-hydroxyapatite composites, biphasic calcium phosphate nanocomposites (Ramay et al., 2004) or ceramic-biodegradable polymer composites for the use in bone repair.

A suitable synthetic composite implant may achieve properties, which cannot be attained in either of the components materials. Ideally, such a composite should combine the bone-bonding potential of calcium phosphates and excellent biocompatibility with the dynamic mechanical properties of the polymeric components. Several groups have formed composite structures by either mixing polymer with ceramic powders including hydroxyapatite and tricalcium phosphate or precipitating an apatite-like layer on the polymer surface (U.S. Pat. No. 5,766,618; U.S. Pat. No. 6,165,486; U.S. Pat. No. 6,281,257; U.S. Pat. No. 6,867,240; Guan et al., 2004; Ramay et al., 2003; Kim et al., 2004). Kim et al. have prepared poly(epsilon-capronolactone) (PCL) and biphasic calcium phosphate composite membranes (films) by a solvent casting method as drug delivery system for an antibiotic (Kim et al., 2004). Others have formed Polymer/ceramic composite scaffolds based on microsphere technology by a unique approach that involves synthesizing calcium phosphate within the forming microspheres (U.S. Pat. No. 5,766,618). Guan et al. developed a scaffold fleece with a porosity over 80%, but very low mechanical strength with the disadvantages of the manufacturing process described for leaching below (Guan et al., 2004).

The employ of basic calcium phosphates in these composite materials can balance the local pH value when the polymer undergoes degradation into acetic monomers to keep a constant physiological pH-value at the defect site (Schiller et al., 2003).

The methods used for the production of composites were based on mixing the ceramics with monomers before polymerization, mixing polymer solutions with ceramics and subsequent drying or by cold and hot pressing powder mixtures. Further processes to prepare biodegradable polymer materials are described in U.S. Pat. No. 6,436,426, hereby incorporated as reference. The processing of such composites often requires thermal treatment or high sintering temperatures and the use of solutions such as chloroform for the polymeric component (Ignjatovic et al., 1999; Durucan et al., 2000; Ramay et al., 2004). These processing methods are not applicable if the material has to be combined with a sensitive osteoinductive protein due to the degradation and instability of the protein.

Furthermore these processing steps often yields to a dense material were a suitable porosity has to be introduced via water-soluble crystals (e.g., salt leaching) in a subsequent process step were the material is incubated in water. During this procedure it is most likely that the protein is also dissolved or undergoes degradation. In addition, not a fully porous scaffold throughout the matrix will be generated limiting cell infiltration and new bone generation.

### Biomaterials and Osteoinductive Proteins

To achieve an osteoinductive effect an alternative to the use of autogenic bones is the use of specific bone growth and differentiation factors such as GDF-5 or different bone morphogenetic proteins (BMPs). Numerous animal studies clearly show that this osteoinductive effect can be greatly increased if these protein factors are combined with a carrier which decelerated the protein release and therefore increased the effective residence time of the protein at the defect site and finally to accelerate bone-healing compared to liquid formulation buffers (Seeherman et al., 2003). In the literature, calcium phosphates, collagen, mineralised collagen (collagen-containing calcium phosphate) and bioresorbable polymers are described as carriers (hydroxyapatite and beta-TCP (Hotz et al., 1994), hydroxylic apatite from algae extracts (Gao et al., 1996), bone extracts (Gombotz et al., 1996), collagen (Friess et al., 1999) and poly(alpha-hydroxy acids) (Hollinger et al., 1996).

The analyses of the potency of the coated carriers, which are described in the literature, do not present a uniform picture but exhibit significant variations which are a consequence of either the carrier type selected or the coating method (Terheyden et al., 1997). Various methods are described.

In WO 98/21972 coating by rapid precipitation of GDF-5 onto beta-TCP is achieved by first dissolving GDF-5 in an organic solvent and then precipitating it by adding water. Due to the toxicity of many solvents, however, such a process is not preferred for the production of pharmaceutical compositions. Lind et al. (1996) carry out the coating of various calcium phosphate ceramics in the presence of gelatine (usually obtained from bovine or pig bones) as protection protein. Due to the increased risk of infection and immunogenic reactions, however, the use of animal substances should be avoided for the production of pharmaceutical compositions and medicinal products. Friess et al. (1999) and Gao et al. (1996) describe the coating of collagens with BMP-2. Due to the low compressive strength of collagens, such carriers, however, are not suitable for many indications. This particularly applies to indications with which the newly-formed bone has to sustain a later pressure load. Furthermore, pharmaceutical qualities of collagen are so far available from animal sources only. Finally, according to the fast degradation rate and release of the growth factors in the state of the art products (e.g. rhBMP-2 and collagen sponge) the drug substance content is often dramatically above the physiological level in the bone tissue.

Advantageously, as disclosed in WO 03/043673, it has been found by the present inventors that improved and reliable osteoinductive and osteoconductive properties in vivo after implantation into a subject, preferably a human, is achieved in a device, wherein a homogenous distribution of the composite carrier, such as beta-TCP or other calcium phosphates, with biologically active, non-aggregated osteoinductive protein can be realized. Such aggregation causes micro-precipitation, which is the reason for an inhomogenous distribution resulting in at least significantly decreased osteoinductive properties as described for other devices in the prior art, e.g., in WO98/21972. Moreover, it has been found that undesirable side effects, such as inflammation and toxic reactions of the subject after implantation, can be avoided by the device of the present invention, which is free of toxic impurities or infectious contaminants. In particular, the use of protecting proteins (such as e.g. gelatine) as solubility mediator is totally unnecessary for the device of WO 03/043673. However, such devices are not suitable for applications requiring a retarded release of the active agent.

In the field of bone augmentation retarded release systems are especially required in view of short half-life of proteins or peptides in the human body with respect to bone induction, either due to dispersion from the implant site or through degradation. In first attempts to achieve a retarded release of bone morphogenic proteins, devices have been disclosed, wherein such proteins have been combined with bioresorbable polymers. Hollinger et al. (1996) published the use of poly(alpha-hydroxy acids) as carriers for BMP-2. In combination with osteogenic proteins or peptides these polymers are of special interests with regards to achieve a controlled release of the active agent. Wang et al. (2000) disclose an emulsion freeze-drying process starting with a PLA solution in methylene chloride for the fabrication of a biodegradable scaffold capable of incorporating and delivering bioactive macromolecules for bone regeneration. Schmidmaier et al. (2000) disclose the use of a chloroform solution of PLA together with the osteoinductive factors IGF-I and TGF-beta1 in the coating implants.

WO02/070029 discloses a porous beta-TCP matrix, which is optionally admixed with PLGA microspheres encapsulated with OP-1 (osteogenic protein 1, a bone morphogenic protein) to form a heterogeneous material. In contrast to WO 03/043673 the beta-TCP matrix in WO02/070029 exhibits single separate voids instead of interconnected pores. The pores of this matrix are not capable to be equipped with a homogeneous coating of the polymer and/or active agent component. The microspheres are produced by Alkmeres, Inc and exhibit a 20 to 500 μm diameter permitting microaggregation of the encapsulated active agent. For the production of such microspheres methylene chloride solutions of the polymeric component together with the protein are sprayed and frozen in a deeply cold ethanol (Herbert et al. 1998 and see e.g. U.S. Pat. No. 6,726,860). Both steps in combination with two different organic solvents impart the chemical and mechanical stress to the protein.

In contrast to the also flowable CPC, were the protein is within or onto the carrier in direct contact with the surrounding medium, a protein within a polymer containing carrier (e.g., poly(alpha-hydroxy acids) can be protected and/or stabilized. Furthermore, the protein or peptide is released only by diffusion from the calcium phosphate cement whereas the protein or peptide within the polymer matrix is released with the increasing degradation of the polymer and/or by diffusion from the polymer matrix. Therefore the release kinetic can be fine-tuned more easily than it's the case for the pure calcium phosphate cement.

These compositions comprise of a water insoluble biodegradable polymer in a biocompatible water miscible organic solvent for forming a biodegradable solid implant in situ within the body by exposure to body fluids or aqueous fluid and are administered as liquids using a syringe to form in situ a solid matrix by dissipation or dispersion of the organic solvent within the body. During contact with water a scaffold with a high porous inner core structure surrounded by a nearly none porous surface is formed.

This none porous surface inhibit cell migration into the inner core therefore these material exhibits no osteoconductive properties. These implants are used as prosthetic devices and/or controlled delivery system for biological active agents not sufficient for applications such as bone augmentation.

Another drawback of this type of material class is the prolonged setting time until the material shows a sufficient mechanical stability. However, the subsequent in vivo degradation of the polymer causes similar problems as described above for conventional polymer based scaffolds. They exhibit degradation, leading to a loss in mechanical properties, and a lowering of the local pH to a cytotoxic level. As a consequence this can lead to an inflammatory foreign body response.

In addition, they do not possess the same bioactive and osteoconductive properties of calcium phosphate systems described below.

Up to now there exist no suitable processing technique for the manufacturing of larger mechanically stable porous specimen made from PLGA/calcium phosphate composites designed to incorporate sensitive molecules like proteins or peptides and therefore no material to fulfil the requirements mentioned above.

Therefore there is still need for a technique to incorporate process sensitive molecules into scaffolds or solid three dimensional specimens without process induced protein or peptide degradation and a need for materials obtained with such techniques.

Accordingly, an object underlying the present invention is the provision of a material/device which is suitable for implantation into a subject in the need of bone augmentation, which material allows a retarded release of an attached active agent and preferably further optimized local activity of an enclosed active agent as well as bioresorption.

Another object underlying the present invention is the provision of a material/device, preferably free flowing granules or a composite three dimensional material which is macroporous and/or, suitable for implantation into a subject in the need of bone augmentation allowing retarded release of an attached active agent and avoiding the problems associated with a local pH decrease induced by polymer degradation.

Another object underlying the present invention is the provision of a material/device, preferably free flowing granules or a solid three dimensional material which is macroporous and/or, suitable for implantation into a subject in the need of bone augmentation allowing retarded release of an attached active agent and avoiding toxic side effects and/or inflammatory responses.

Another object underlying the present invention is the provision of a material/device, preferably free flowing granules or a solid three dimensional material which is macroporous and/or, suitable for implantation into a subject in the need of bone augmentation allowing retarded release of an attached active agent and allowing lower doses of the active agent compared to conventional devices.

Another object underlying the present invention is the provision of a material/device, preferably a solid three dimensional material which is macroporous and/or, suitable for implantation into a subject in the need of bone, allowing retarded release of an attached active agent and having the manifestation of a load bearing three-dimensional implant with mechanical properties preferably similar to cancellous or trabecular bone.

### SUMMARY OF THE INVENTION

Surprisingly, the present inventors were able to provide a material solving these objects and corresponding methods for the production of said material.

Thanks to the present invention the inventors could provide composite materials preferably free flowing granules or macroporous and/or microporous solid three dimensional scaffolds, preferably with the manifestation of a load bearing three-dimensional implant with mechanical properties preferably similar to trabecular bone.

Furthermore, the present inventors provide a method for producing a composite material comprising of a water insoluble solid filler and an active agent homogenously dispersed within the polymer or homogeneously coated on the filler, wherein the polymer is solved in a solution which relates to a pharmaceutical acceptable organic solvents capable to dilute the polymer and compatible with the active agent, comprising the steps of freeze drying and thermal treating preferably under vacuum.

Preferably the composite materials of the present invention are solvent free.

The term “solvent free” refers to a composite comprising a water insoluble polymer and a water insoluble solid filler, preferably calcium phosphate, wherein the interstices of said matrix are substantially free from residual solvent such that said composite material reaches a constant mass upon evaporation.

By the term “substantially free” it is meant that, with normal detection methods such as detection by changes in mass, no solvent is detected. While it is believed that the composite material is completely free of solvent, it is possible that extremely small quantities might be measurable by highly sensitive analytical methods. By using the method of the invention, selection of a suitable solvent, freeze drying and thermal treatment of said composite material preferably said solvent free composite material can be manufactured which enable generation of an active agent containing composite material with improved efficacy, retarded release of the active agent and/or reduced amount of protein degradation.

Accordingly, the present invention provides the following embodiments:

### Embodiments

1. Sterile pharmaceutical acceptable free flowing granules of a composite material comprising
   a) a water insoluble solid filler, preferably a beta-tricalcium phosphate,
   b) a water insoluble polymer, preferably a PLGA, and
   c) an active agent homogenously dispersed within the polymer or homogeneously coated on the filler,
   wherein the content of the intact active agent is equal to or more than 70%, preferably 80%, most preferably 90%.
2. The sterile free flowing granules of Embodiment 1, wherein the polymer is homogeneously coated on the filler.
3. A sterile composite 3-dimensional scaffold comprising
   a) a water insoluble solid filler, preferably a beta-tricalcium phosphate,
   b) a water insoluble polymer, preferably a PLGA, and
   c) an active agent homogenous dispersed within the polymer, or homogeneously coated on the filler,
   wherein the content of the intact active agent is equal to or more than 70%, preferably 75%, most preferably 80%.
4. The composite 3-dimensional scaffold of Embodiment 3, which is microporous,
   wherein the polymer to carrier ratio of the material is between 0.15 and 1
   and the scaffold is obtained using a carrier comprising beta-tricalcium phosphate powder as educt.
5. The composite 3-dimensional scaffold of Embodiment 3, which is macroporous,
   wherein the polymer to carrier ratio of the material is between 0.2 and 0.67
   and the scaffold is obtained using a carrier consisting of beta-tricalcium phosphate granules, preferably with an average diameter of greater than 0.1 mm, more preferably between 0.5 and 4 mm, as educt.
6. The composite 3-dimensional scaffold of Embodiment 4,
   wherein the polymer to carrier ratio is between 0.2 and 1, preferably between 0.33 to 1, most preferably 0.65 to 0.67 and the polymer content not more than 50 wt %, preferably less than 45 wt %, most preferable between 30-40 wt % wherein the composite material has a compressive strength between 5 and 65 MPa and a Young's modulus of 15 to 30 MPa.
7. The composite 3-dimensional scaffold of Embodiment 5,
   wherein the polymer carrier ratio is between 0.25 and 0.67, preferably between 0.45 and 0.56 and the polymer content not more than 35 wt %, preferably between 15-35 wt % wherein the composite material has a compressive strength between 1 and 10 MPa and a Young's elastic module of 9 to 55 MPa.
8. The sterile free flowing granules of Embodiments 1 to 2 or the composite 3-dimensional scaffold of Embodiments 3 to 7, wherein the release rate of the active agent from the free flowing granules and the composite 3-dimensional scaffold is a sustained release rate.
9. The sterile pharmaceutical acceptable free flowing granules of Embodiments 1 to 2 or the composite 3-dimensional scaffold of Embodiments 3 to 8, wherein the water insoluble solid carrier contains a calcium phosphate selected from beta tricalcium phosphate, alpha tricalcium phosphate, apatite and a calcium phosphate containing cement or a mixture of them.
   Most preferred is beta tricalciumphosphate.
10. The sterile pharmaceutical acceptable free flowing granules or the composite 3-dimensional scaffold of Embodiment 9, wherein the water insoluble polymer is biodegradable, biocompatible, and/or bioresorbable.
   Preferably the water insoluble polymer is a poly(alpha-hydroxy acids), poly(ortho esters), poly(anhydrides), poly(aminoacids), polyglycolids (PGA), polylactids (PLLA), poly(D,L-lactide) (PDLLA), poly(D,L-lactide co-glycolide) PLGA), poly(3-hydroxybutyricacid) (P(3-HB)), poly(3-hydroxy valeric acid) (P(3-HV)), poly(p-dioxanone) (PDS), poly(epsilon-caprolactone) (PCL), polyanhydride (PA), polyorthoester, polyethylene (PE), polypropylene (PP), polyethyleneterephthalate (PET), polyglactine, polyamide (PA), polymethylmethacrylate (PMMA), polyhydroxymethylmethacrylate (PHEMA), polyvinylchloride (PVC), polyvinylalcohole (PVA), polytetrafluorethylene (PTFE), polyetheretherketone (PEEK), polysulfon (PSU), polyurethane or polysiloxane or a mixture of them.
11. The sterile pharmaceutical acceptable free flowing granules or the composite 3-dimensional scaffold of Embodiment 10, wherein said water insoluble polymer is PLGA, preferable PLGA of a glycolic acid content between 0 to 70 mol %, most preferably a PLGA (50:50) with an inherent viscosity of 0.1 to 0.4 dl/g, preferably 0.1 to 0.3 dl/g, wherein the inherent viscosity is determined at 25° C. and 0.1% solution in chloroform.
12. The sterile pharmaceutical acceptable free flowing granules or the composite 3-dimensional scaffold of Embodiment 10 and 11, wherein the active agent is an osteoinductive polypeptide (protein or peptide).
13. The sterile pharmaceutical acceptable free flowing granules or the composite 3-dimensional scaffold of Embodiment 12, wherein said osteoinductive polypeptide is a member of the TGF-beta family, preferably a member of the BMP family.
   Details regarding osteoinductive polypeptides incorporated in the sterile pharmaceutical acceptable free flowing granules and the composite 3-dimensional scaffold of the present invention are described below under the corresponding method embodiment, in particular embodiments 30 to 34, and apply to product embodiments as well.
14. A method for the production of a composite material comprising the steps of:
   (a) providing an aqueous solution comprising an active agent and a buffer, which buffer keeps said active agent dissolved for a time sufficient to allow homogenous coating of a carrier, preferably a ceramic carrier when said carrier is contacted with said solution;
   (b) contacting the solution of step (a) with a water insoluble solid carrier, preferably a ceramic carrier, more preferably a ceramic carrier containing calcium phosphate;
   (c) allowing homogenous coating of the surface of said water insoluble solid carrier with said dissolved active agent;
   (d) drying the coated water insoluble solid carrier obtained in step (c);
   (e) providing a further solution comprising a dissolved water insoluble polymer or a mixture of water insoluble polymers, which polymer stays dissolved for a time sufficient to allow homogenous coating of the water insoluble solid carrier obtained in step (d) when said water insoluble solid carrier is contacted with said solution, wherein the water insoluble solid carrier and the active agent coated onto said water insoluble solid carrier is not soluble in said solution;
   (f) freeze drying the polymer coated carrier obtained in step (e); and
   (g) thermally treating said polymer coated carrier obtained in step (f), preferably under vacuum.
   This first method (“method A”) is suitable for an active agent insoluble in an organic polymer solution.
   In a further preferred embodiment the method A above includes an additional step of closing the packaging container with the composite material after thermal treatment to ensure an inert atmosphere to improve the long time stability of the active agent and therefore of the final product.
15. A method for the production of a composite material comprising the steps of:
   (a) providing a solution comprising an active agent, and a water insoluble polymer or mixture of water insoluble polymers;
   (b) contacting the solution of step (a) with a water insoluble solid carrier, preferably a ceramic carrier, more preferably a ceramic carrier containing calcium phosphate,
   (c) allowing homogeneous coating of the surface of said carrier with said dissolved active agent and polymer
   (d) freeze drying the polymer coated carrier obtained in step (b); and
   (e) thermally treating said coated carrier obtained in step (d), preferably under vacuum.
   This second method (“method B”) is suitable for an active agent soluble or suspensible (i.e. compatible) in the organic polymer solution.
   In a further preferred embodiment the method B above includes an additional step of closing the packaging container with the composite material after thermal treatment to ensure an inert atmosphere to improve the long time stability of the active agent and therefore of the final product.
   In a further preferred embodiment the method A or B of the present invention further contains the addition of fibers such as PGA, PLA, nylon, inorganic fibers, e.g., glass fibers to increase the mechanical properties of the composite material preferably the composite 3-dimensional scaffold. Preferably the fibers are added into the solution of Embodiment 14 (e) and 15 (a) or (b).
16. The method of Embodiments 14 or 15, wherein the solution of Embodiment 14 (e) and Embodiment 15 (a) is a pharmaceutical acceptable organic solvent in which the polymer is soluble, which is compatible with the active agent, which is dryable under reduced pressure and removable by freeze drying.
17. The method of any one of Embodiments 14 to 16, wherein said solution of Embodiment 14(e) and Embodiment 15(a) contains as pharmaceutical acceptable organic solvent a compound selected from anisole, tetramethylurea, acetic acid, dimethylsulfoxide and tert-butanol, 1-butanol, 2-butanole, butyl acetate, tert-butylmethyl ether, cumene, dieethylether, ethylformate, formic acid, 3-methyl-1-butanol, methylethyl ketone, methylisobutylketone, 2-methyl-1-propanole, and 1-methyl-2-pyrrolidone.
18. The method of any of Embodiments 14 to 17, wherein said water insoluble solid carrier contains a calcium phosphate selected from beta tricalcium phosphate, alpha tricalcium phosphate, apatite and a calcium phosphate containing cement.
19. The method of any one of Embodiments 14 to 18, wherein said water insoluble polymer is biodegradable, biocompatible, and/or bioresorbable.
20. The method of any one of Embodiments 14 to 19, wherein said water insoluble polymer is selected from a poly(alpha-hydroxy acids), poly(ortho esters), poly(anhydrides), poly(aminoacids), polyglycolids (PGA), polylactids (PLLA), poly(D,L-lactide) (PDLLA), poly(D,L-lactide co-glycolide) PLGA), poly(3-hydroxybutyricacid) (P(3-HB)), poly(3-hydroxy valeric acid) (P(3-HV)), poly(p-dioxanone) (PDS), poly(epsilon-caprolactone) (PCL), polyanhydride (PA), polyorthoester, polyethylene (PE), polypropylene (PP), polyethyleneterephthalate (PET), polyglactine, polyamide (PA), polymethylmethacrylate (PMMA), polyhydroxymethylmethacrylate (PHEMA), polyvinylchloride (PVC), polyvinylalcohole (PVA), polytetrafluorethylene (PTFE), polyetheretherketone (PEEK), polysulfon (PSU), polyethyleneglycol (PEG), polyvinylpyrolidone, polyurethane or polysiloxane.
21. The method of Embodiment 14 to 20, wherein said water insoluble polymer is PLGA, preferably PLGA of a glycolic acid content between 0 to 70 mol % (m %), preferable 50 mol %, most preferably a PLGA (50:50) with an of 0.1 to 0.4, preferably 0.1 to 0.3 dl/g, wherein the inherent viscosity is determined at 25° C. and 0.1% solution in chloroform.
22. Method of Embodiments 14 to 21, wherein the freeze drying is performed under ambient temperature and thermal treating is performed above the glass transition temperature of the polymer system but below the denaturing temperature of the active agent.
   This allows for a high content of the intact active agent of equal to or more than 70%, preferably 80%, most preferably 90% in sterile pharmaceutical acceptable free flowing granules or the composite 3-dimensional scaffold of embodiments 1 to 13.
23. Method of Embodiment 22, wherein the temperature of the thermal treatment is between 45° C. and 80° C., preferably between 50° C. and 65° C.
24. The method of Embodiments 14 to 23, wherein said biodegradable composite material is formed to exhibit a microporous solid three dimensional scaffold, preferably with the manifestation of a load bearing three-dimensional implant with mechanical properties preferably similar to trabecular bone, wherein the water insoluble carrier in step (b) of Embodiments 14 or 15 comprises a powder form and the polymer content of the material is between 10 and 50 wt %, preferably 30 to 45%, most preferably PLGA (50:50) 30 wt % to 45 wt %.
25. The method of Embodiments 14 to 23, wherein said biodegradable composite material is formed to exhibit a macroporous solid three dimensional scaffold, preferably with the manifestation of a load bearing three-dimensional implant with mechanical properties preferably similar to trabecular bone, wherein the water insoluble carrier in step (b) of Embodiments 14 or 15 consists of a granular form and the polymer content of the material is between 19 wt % and 45 wt % preferably 30 to 45 wt %, most preferably PLGA (50:50) 30 wt % to 45 wt %.
26. The method of Embodiments 14 to 23, wherein said biodegradable composite material is formed to exhibit free flowing granules, wherein the water insoluble carrier in step (b) of Embodiments 14 or 15 consists of a granular form and the polymer content of the material is between 0 wt % and 25 wt %, preferably 0.05 to 20 wt %, even more preferably 0.5 to 20 wt %, or 2 to 20 wt %, more preferably 4 to 20 wt %, or 4 to 15 wt %, 4 to 10 wt %, most preferably 2 to 10 wt %.
   In these embodiments PLGA (50:50) is the preferred water insoluble polymer.
27. The method of any one of Embodiments 14 to 26, wherein said material has osteoinductive and osteoconductive properties in vivo.
28. The method of any one of Embodiments 14 to 27, wherein said active agent is an osteoinductive polypeptide (protein or peptide).
29. The method of Embodiment 28, wherein said osteoinductive polypeptide is a member of the TGF-beta family, preferably a member of the BMP family.
30. The method of Embodiment 29, wherein said member of the BMP family is selected from the group consisting of BMP-1, BMP-2, BMP-3, BMP-4, BMP-5, BMP-6, BMP-7, BMP-8, BMP-9, BMP-10, BMP-11, BMP-12, BMP-13, BMP-14, BMP-15 and BMP-16.
31. The method of Embodiment 30, wherein said member of the TGF-beta family is selected from the group consisting of GDF-1, GDF-2, GDF-3, GDF-4, GDF-5, GDF-6, GDF-7, GDF-8, GDF-9, GDF-10 and GDF-11.
32. The method of any one of Embodiments 14 to 28, wherein said active agent is selected from the group consisting of hormones, cytokines, growth factors, antibiotics, steroids, prostaglandines and other natural or synthesized drug substances.
33. The method of Embodiment 14 to 28, wherein said active agent is parathyroid hormone (PTH) and/or PTH 1-34 peptide.
34. The method of any one of Embodiments 14 to 33, wherein said composite material is biocompatible, biodegradable, and/or bioresorbable.
35. The method of any of Embodiments 14 to 34, further comprising a step of hot pressing after the step of thermally treating.
36. The method of any of Embodiments 14 to 34, further comprising step of filling the polymer coated carrier obtained by step (e) of Embodiment 14 or step (c) of Embodiment 15 in an implant device and prosecuting the respective methods of Embodiments 14 with step (f) and Embodiment 15 with step (d) within the implant device.
37. The method of any of Embodiments 14 to 34, further comprising a step of filling the polymer coated carrier obtained by step (d) of Embodiment 14 or performing step (b) of Embodiment 15 with the water insoluble solid carrier, which has been filled into the implant device, and prosecuting the respective methods of Embodiments 14 with step (e) and 15 with step (c) within the implant device.
   In these Embodiments 35 and 36 a composite material preferably a macroporous and/or microporous composite 3-dimensional scaffold is generated combining the composite material with a further implant device with different structural features (e.g. further increased mechanical stability) to achieve a load bearing outer structure such as a cage and the features as shown in examples 3 and 5. By combining the composite material according to the present invention with a load bearing structure the application of the composite material can be further extended to other indications for example heavy load bearing indications such as spinal fusion, arthrodesis and other long bone defects or fractures.
38. A composite material, which is obtainable by the method of any one of Embodiments 14 to 37.
39. A pharmaceutical composition comprising the composite material of Embodiment 38.
40. Use of the composite material of Embodiment 33, the sterile pharmaceutical acceptable free flowing granules of Embodiments 1 to 3 and Embodiments 8 to 13 and the composite 3-dimensional scaffold of Embodiments 4 to 13 for the preparation of a pharmaceutical composition for bone augmentation, for treating bone defects, degenerative and traumatic disc disease, bone dehiscence for filling cavities and/or support guided tissue regeneration in periodontology.
41. The use of Embodiment 40, wherein said bone augmentation follows traumatic, malignant or artificial defects, sinus floor elevation or augmentation of the atrophied maxillary or mandibular ridge.
42. The use of Embodiment 40, wherein said bone defects are long bone defects, defects in the maxillofacial area or bone defects following apicoectomy, extirpation of cysts or tumors, tooth extraction, or surgical removal of retained teeth.
43. A kit comprising the composite material of Embodiment 38.

The kit might contain the composite material, an application device, such as a syringe, a cylindrical shaped tube with a plunger, a device, a cage, a reconstitution liquid, platelet derived growth factor, platelet enriched plasma, a cutter for shape adjustment, a sterile receptacle, a spatula or a combination thereof.

### BRIEF DESCRIPTION OF THE FIGURES

FIG. 1: Scheme MD05 Retard/Composite Material for Use as Protein Carrier

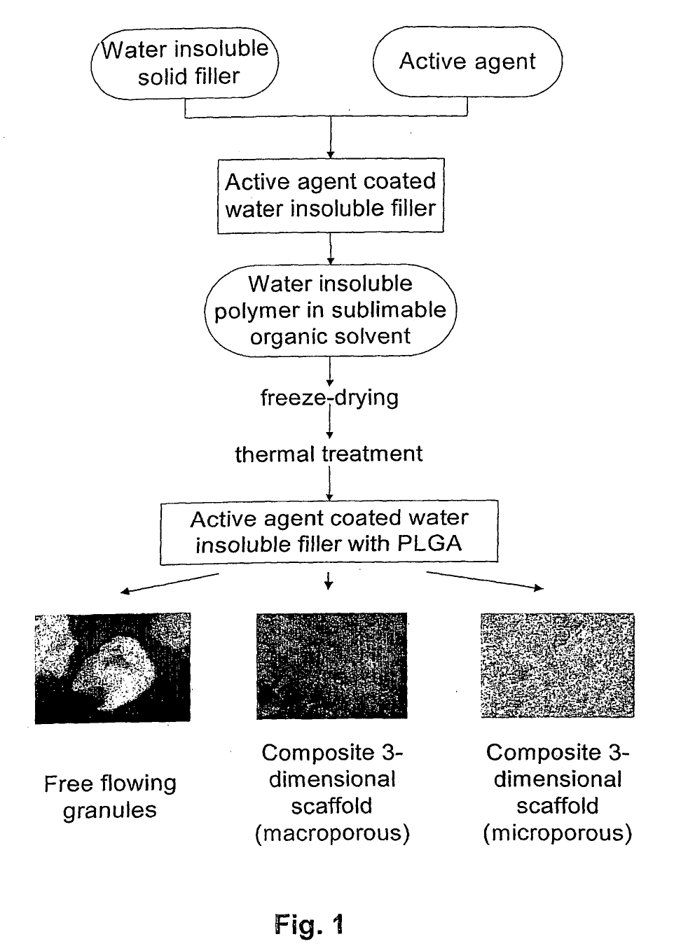

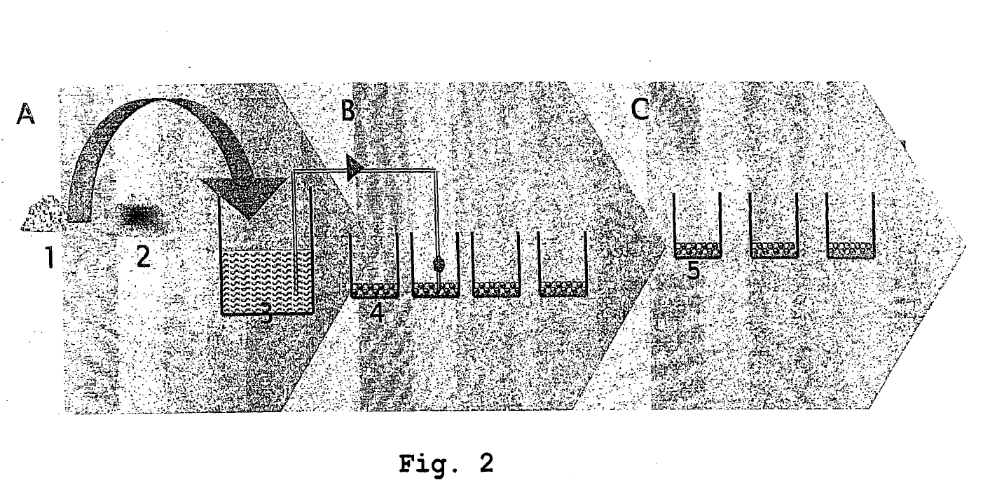

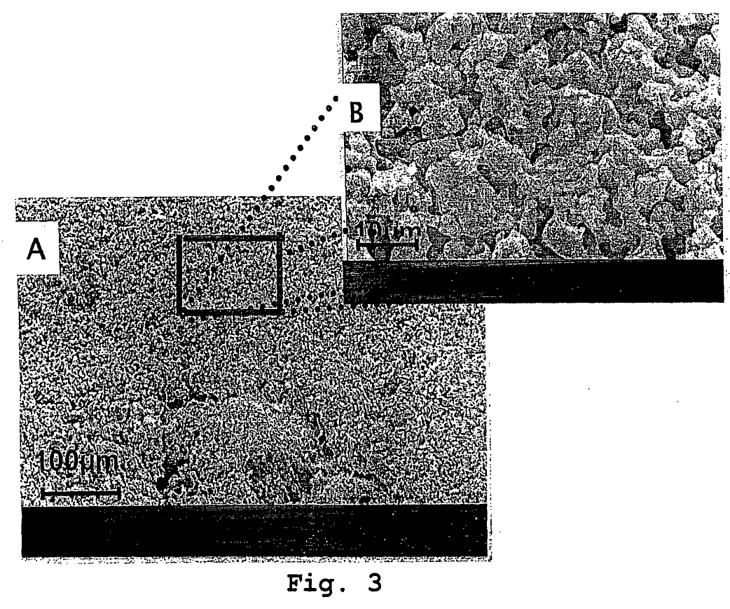

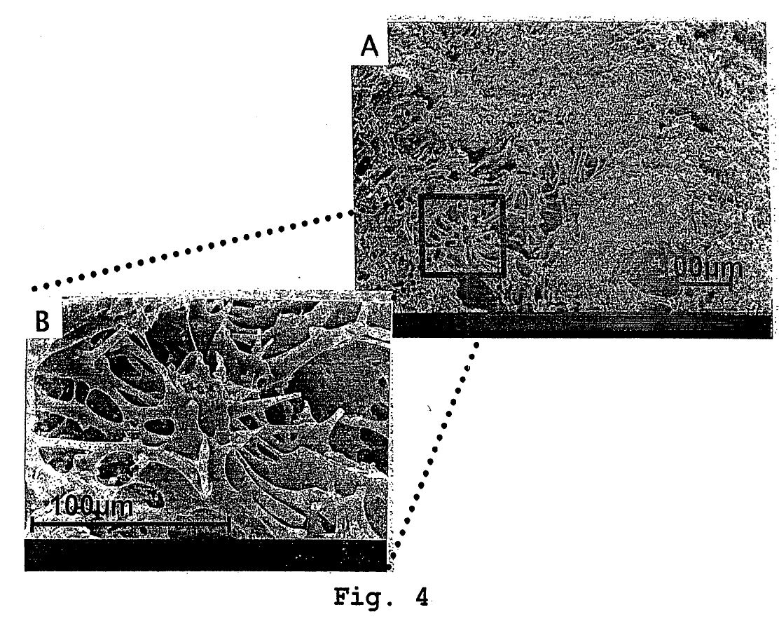

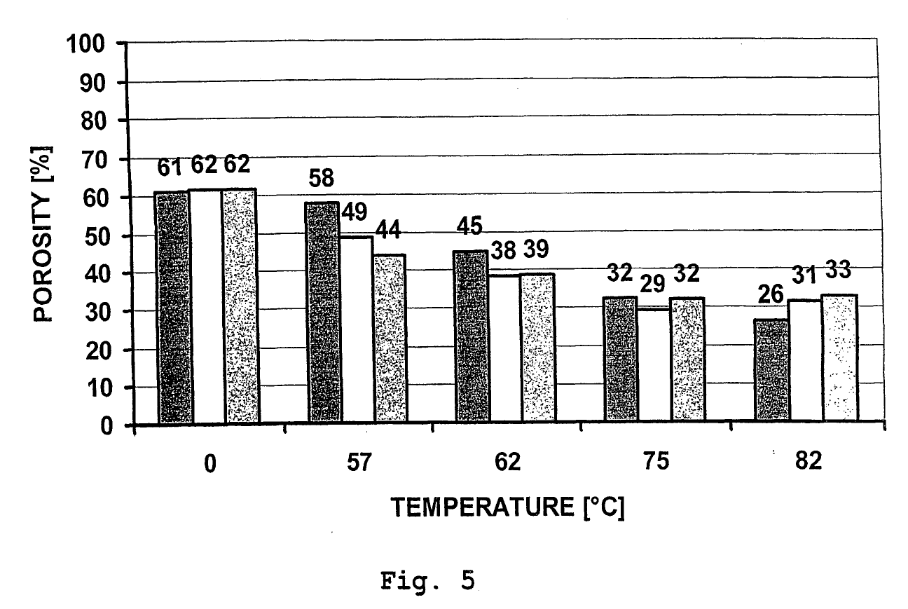

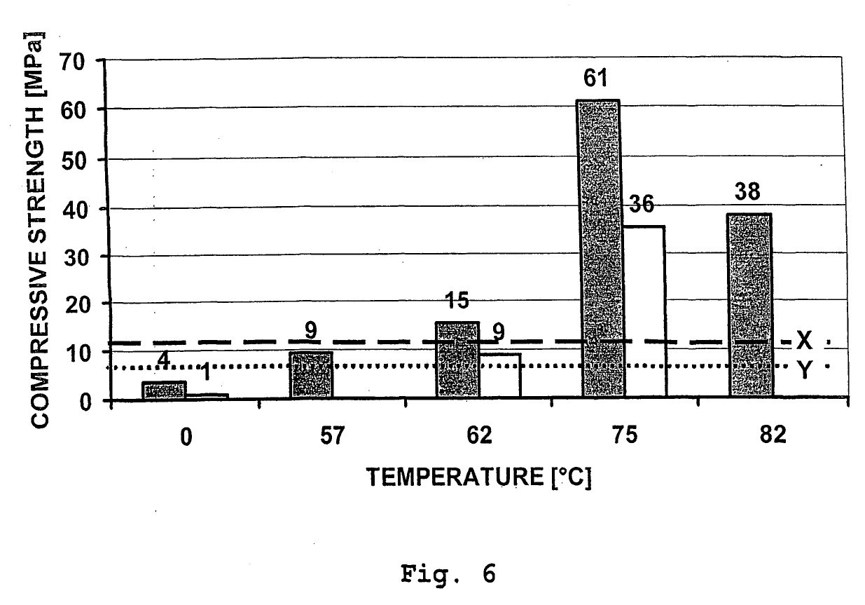

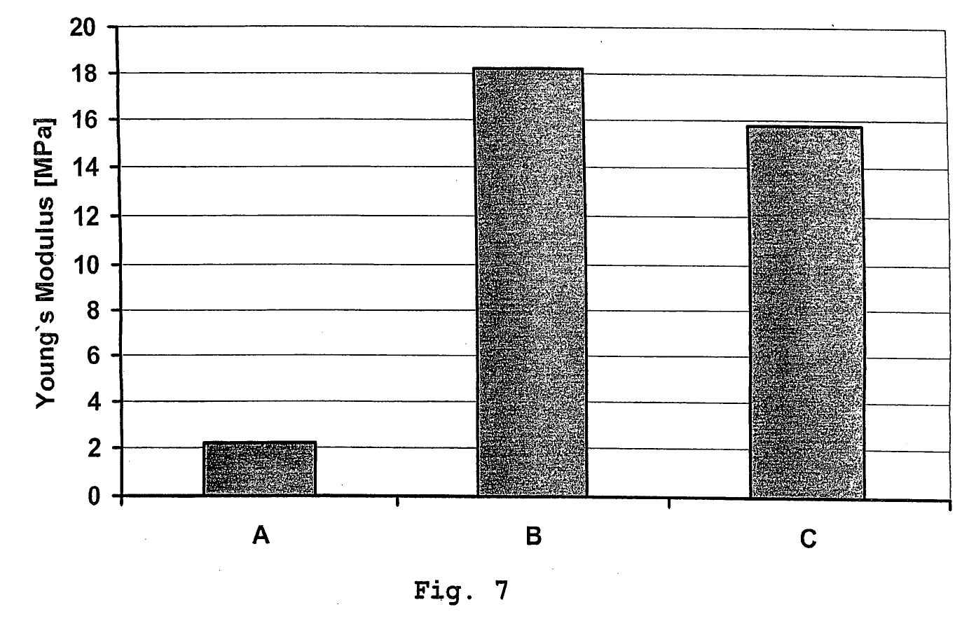

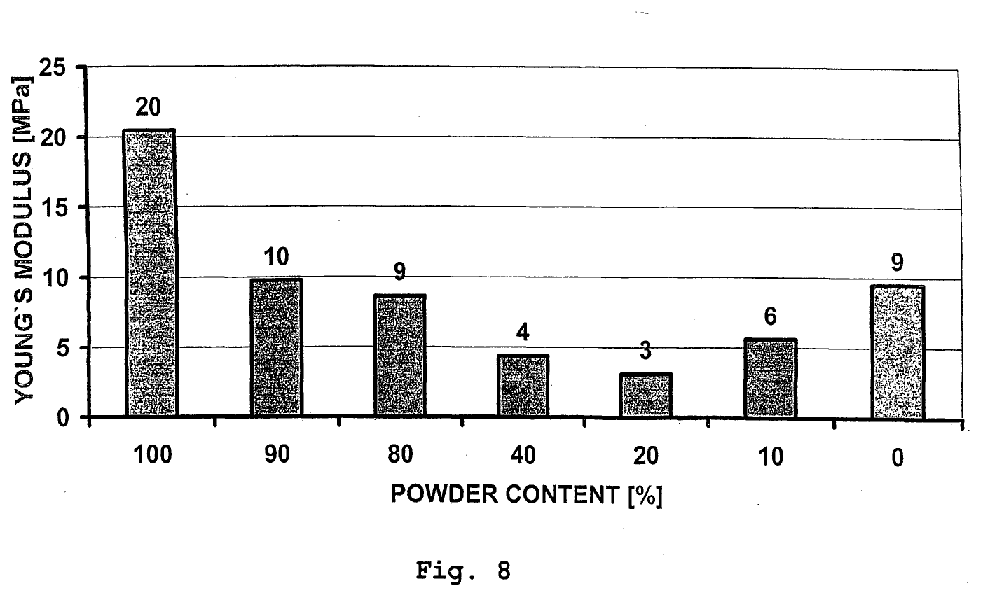

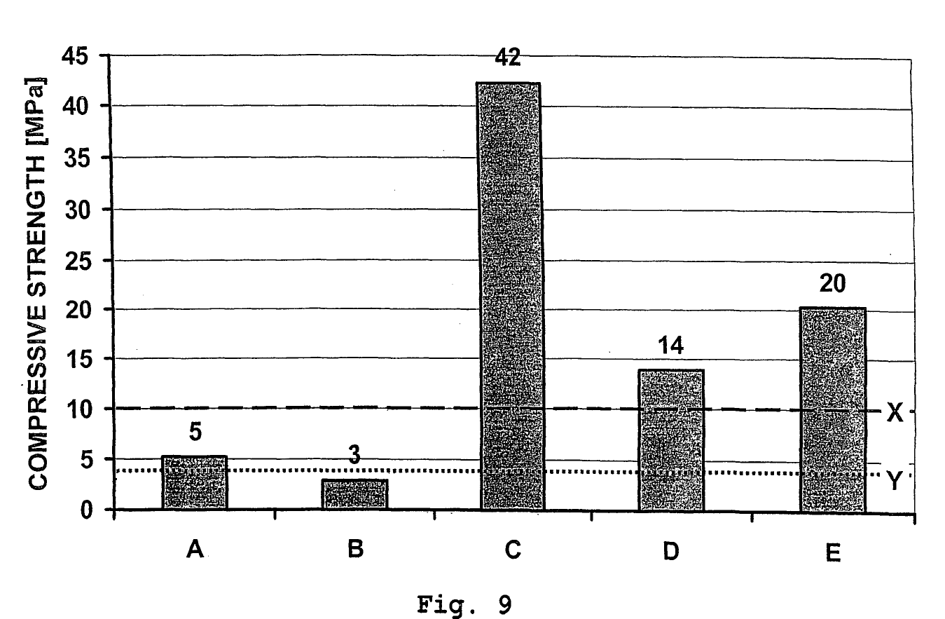

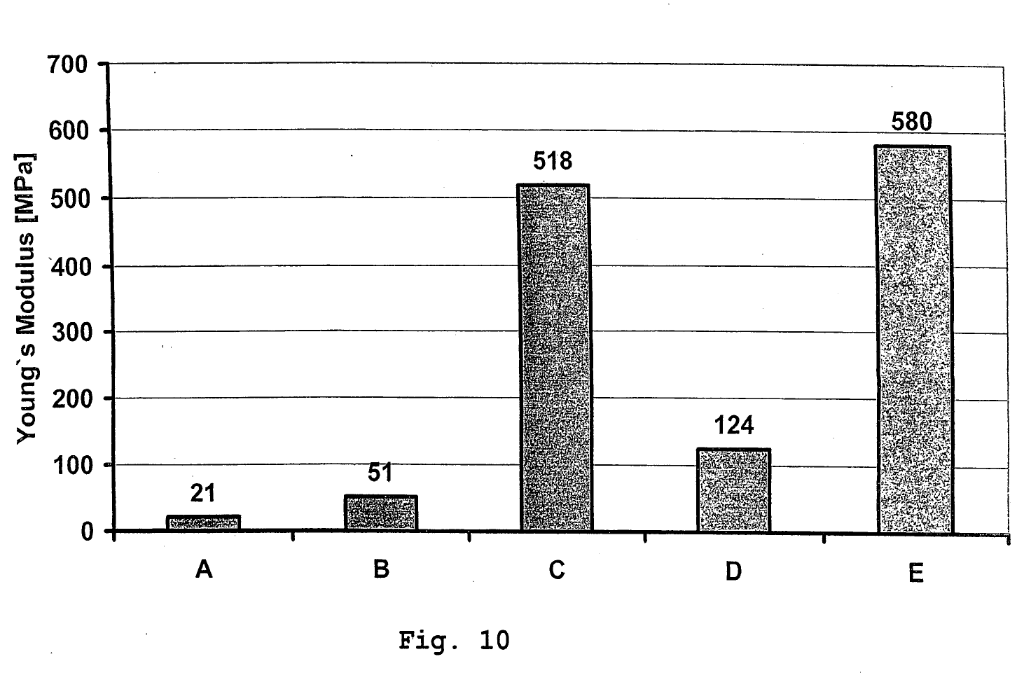

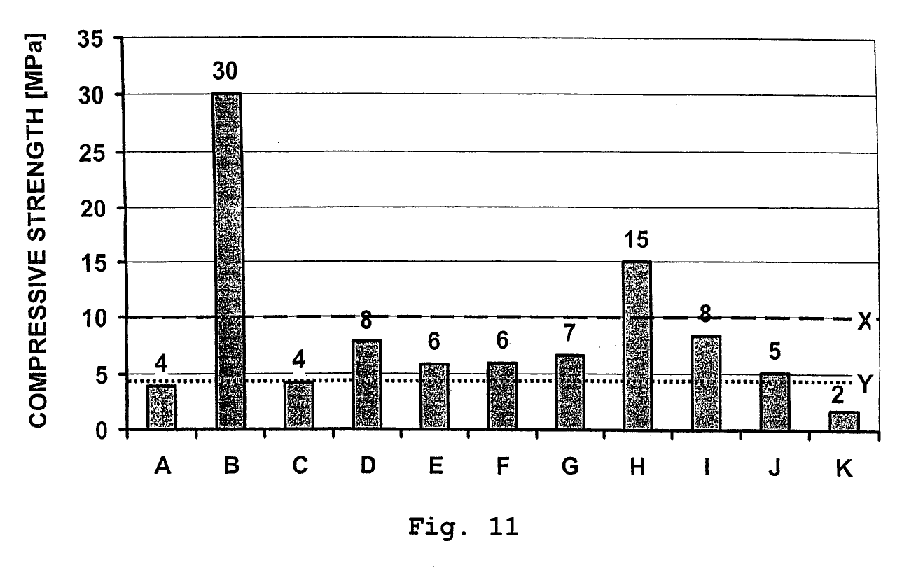

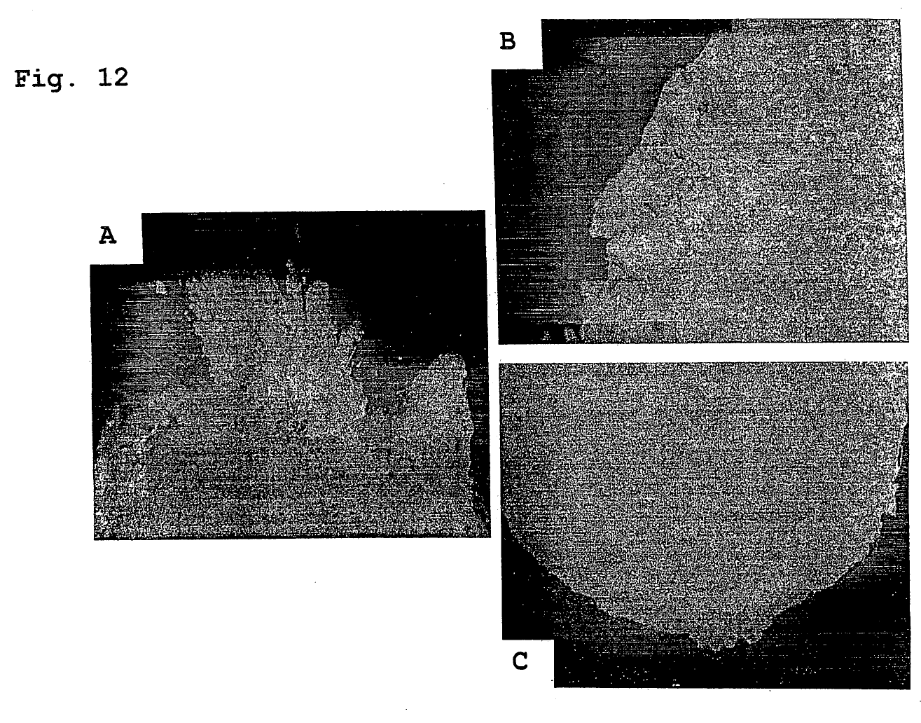

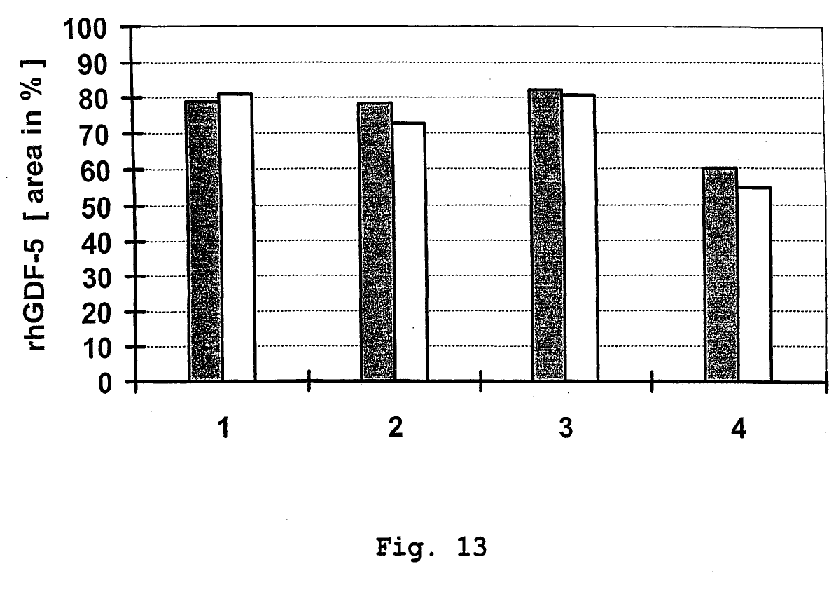

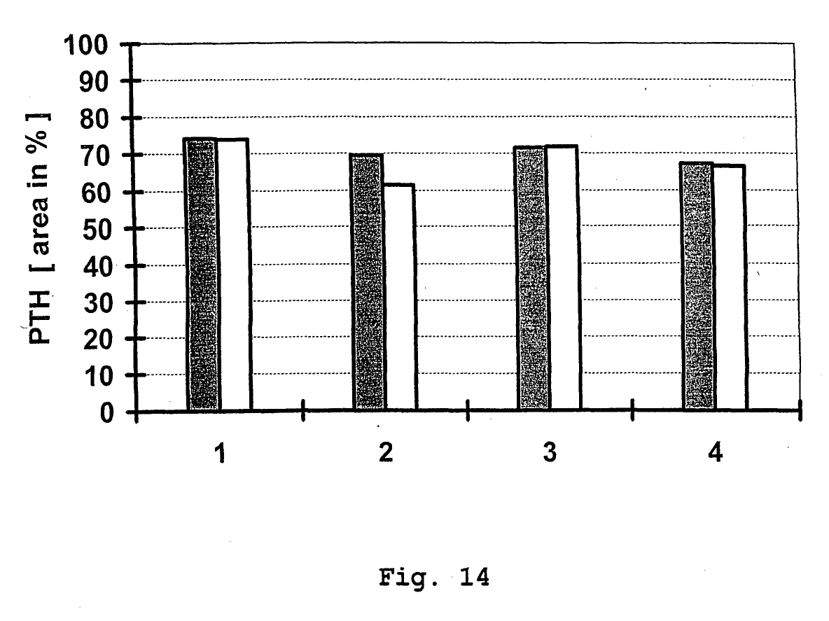

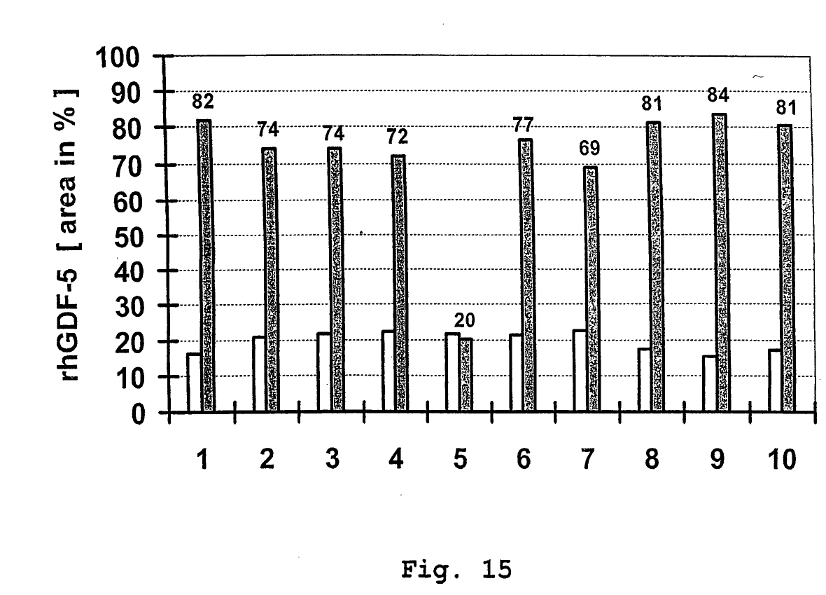

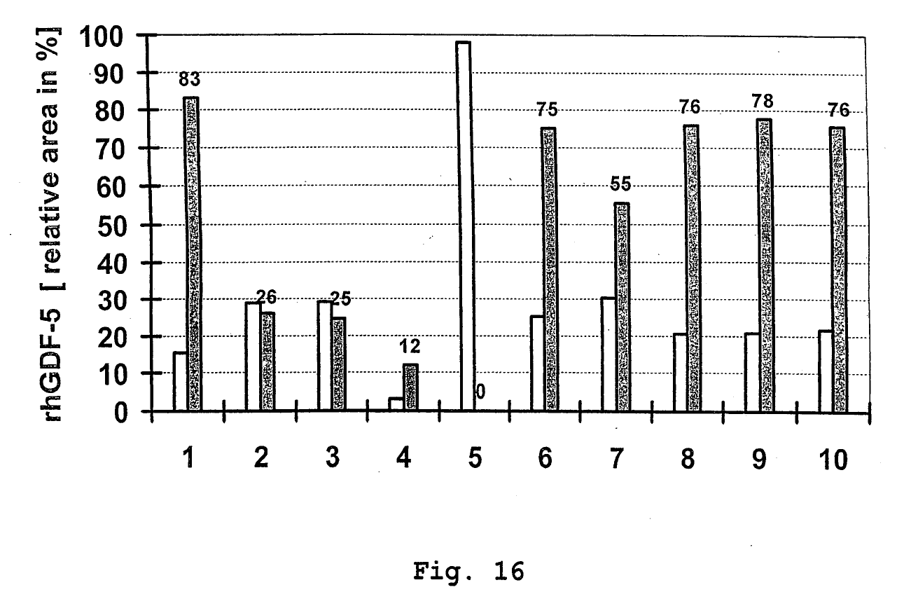

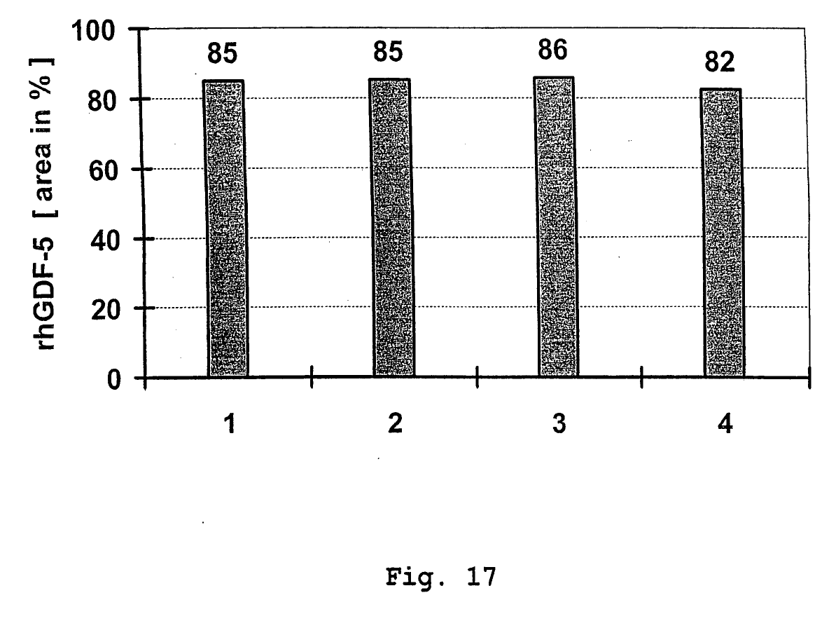

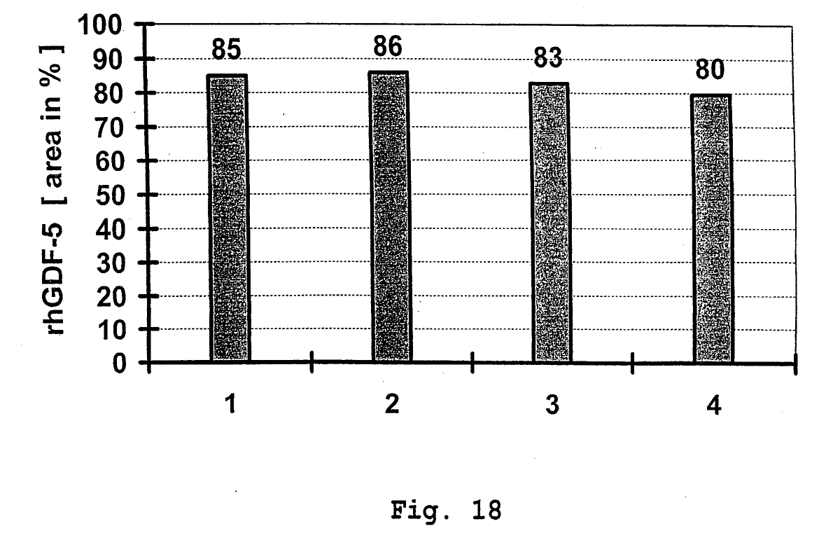

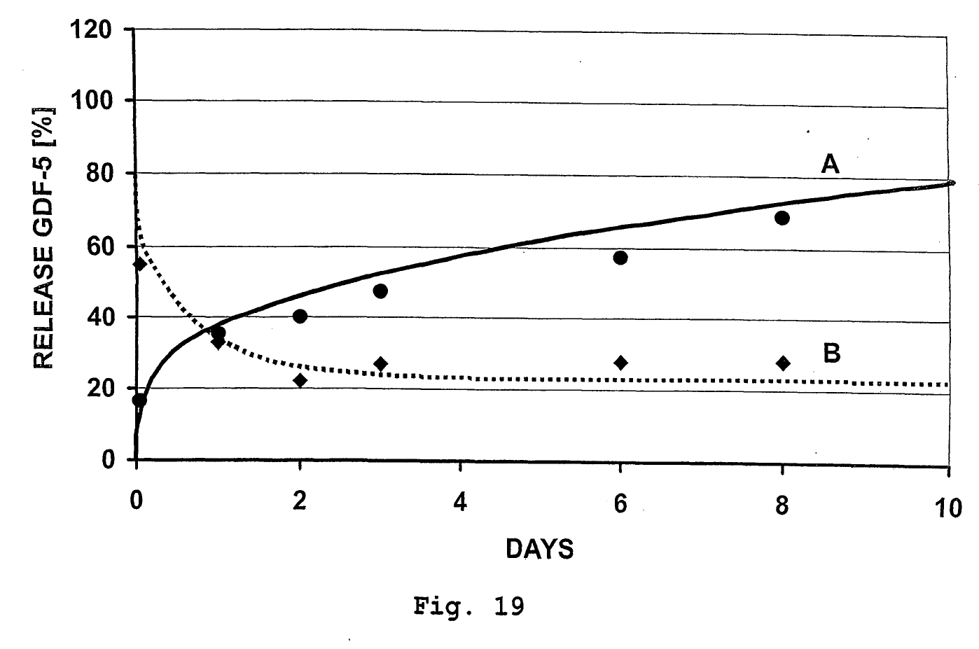

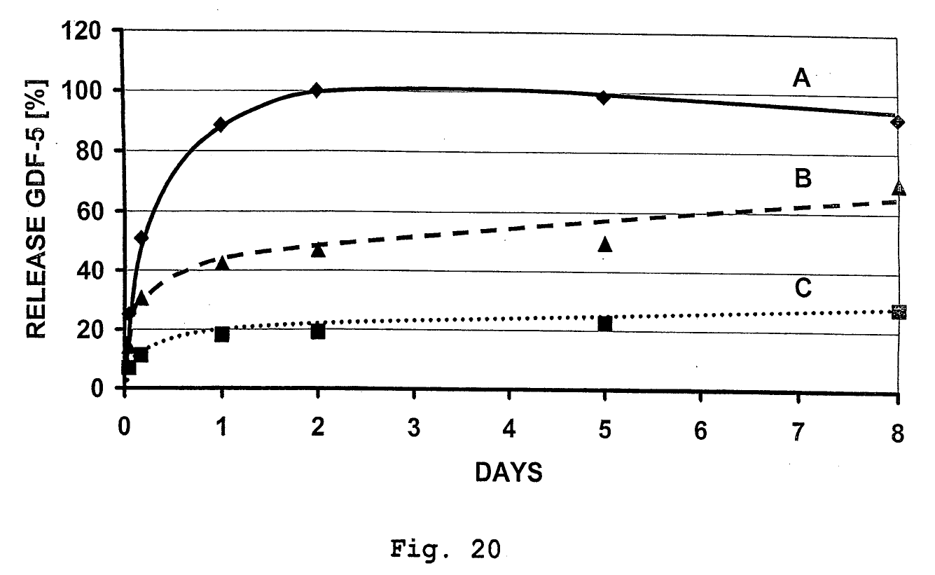

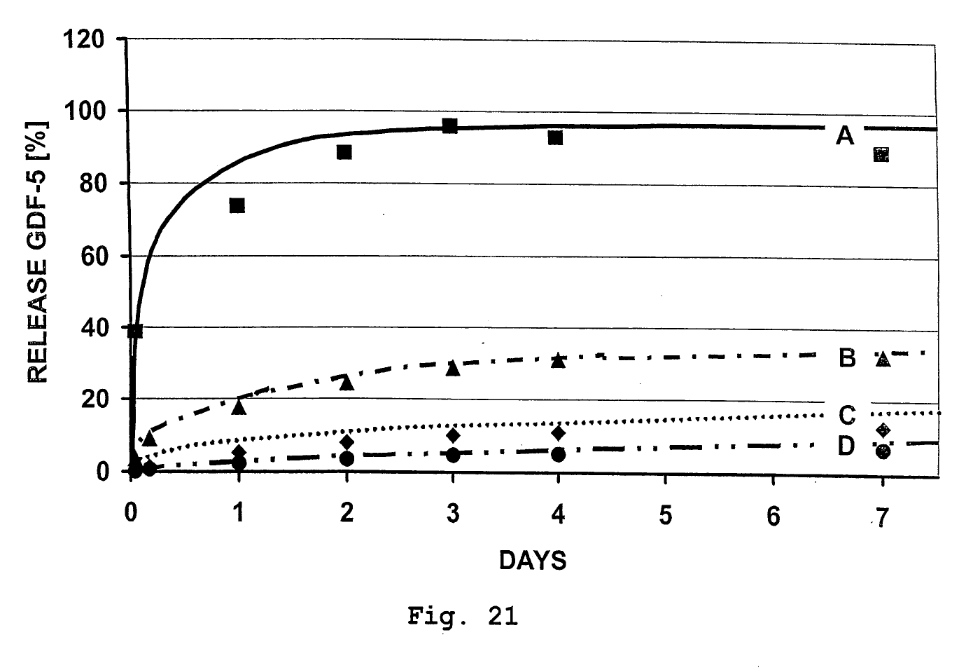

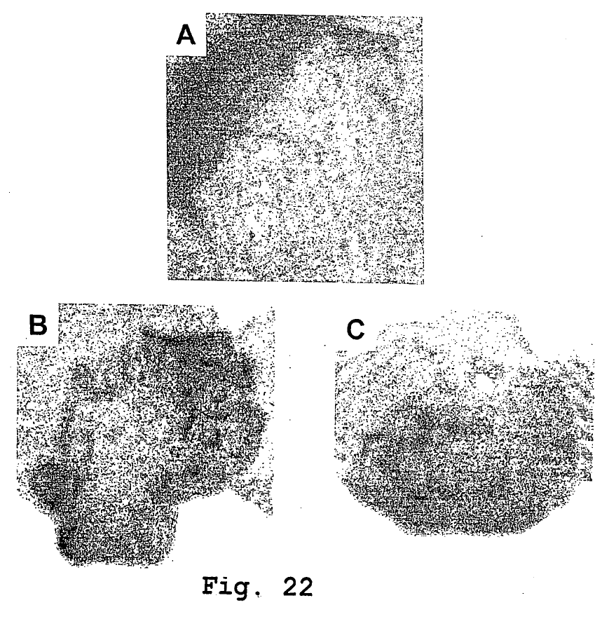

The production process in a preferred embodiment of the present invention (i.e. for calcium phosphate based porous granules with method A) in comparison to the production process set forth in WO 03/043673 (diagram until first rectangular box) is schematically shown. Dependent on the particle size of the starting material (e.g. powder or granules) and the volume and amount of PLGA three different materials can be manufactured using the same manufacturing process: free flowing granules, macroporous composite 3-dimensional scaffold and a microporous 3-dimensional scaffold which is not macroporous.

A water insoluble solid filler in a powder form yields to a microporous composite 3-dimensional scaffold, whereas using granules free flowing granules or a macroporous composite 3-dimensional scaffold is obtained dependent on the polymer to water insoluble solid filler ratio. A further parameter is the volume of the polymer solution. The polymer solution volume has to be in harmony with the density of the inorganic filler to achieve a three dimensional scaffold or free flowing granules as further described in the examples 1 to 6, 18, 19, 22, 23. For example, to achieve a macroporous composite 3 dimensional scaffold the polymer solution volume has nearly the same volume as the mean bulk density of the granules. Higher volumes result in the formation of an inhomogeneous composite material. To achieve free flowing granules the volume of the polymer solution is sufficient to allow complete wetting of said carrier without supernatant liquid of the polymer solution. In general the concentration of polymer within the solution is reduced compared to the manufacturing of the macroporous composite 3-dimensional scaffold. A microporous composite 3-dimensional scaffold is achieved by using a water insoluble solid filler in a powder form in an excess of polymer solution. The upper limit is the viscosity of the polymer filler suspension due to handling properties.

Addition of compounds such as polyglycolide (PGA) fibers, glass, nylon or other fibers can further increase or introduce macroporosity as shown in FIG. 12.

FIG. 2 shows the production of a sterile composite 3-dimensional scaffold (macroporous) by solvent lyophilization and thermal treatment (tempering) containing a protein or peptide, whereas the protein will be released slowly from the composite.

The process flow chart shows the three steps of the manufacturing process:

Step A (compounding): dissolution of the polymer and eventually the active agent in a suitable organic solvent.

Step B (coating): coating the ceramic material with polymer and/or active agent by soaking and subsequent drying.

Step C (lyophilization and thermal treatment): After removal of the solvent the thermal treatment leads to a defined polymeric shell

1—Protein/Peptide

2—PLGA

3—Solvent

4—β-TCP granules

5—sterile composite 3-dimensional scaffold (macroporous)

FIG. 3 represents a scanning electron micoscropy (SEM) of the bottom side of a microporous composite material derived from β-TCP powder and 30% RG502H polymer solution with a PLGA:β-TCP ratio of 1:1.5 (w/w) at 1:200 (A) and 1:2000 (B) magnification, the sample was prepared according to example 4.

FIG. 4 represents a scanning electron micoscropy (SEM) of cross section of a microporous composite material derived from β-TCP powder and 30% RG502H solution with a PLGA:β-TCP ratio of 1:1.5 (w/w) at 1:200 (A) and 1:750 (B) magnification, the sample was prepared according to example 4.

FIG. 5 shows the porosity of the composite material derived from β-TCP powder in relation to the temper temperature for different PLGA polymers of lactic acid:glycolic acid (dark—LR708; PDLLA; polymer composition: 69 mol % L-Lactide and 31 mol % D,L-Lactide; inherent viscosity: 6.0 dl/g, 25° C., 0.1% in CHCl₃; white—RG503H; PLGA; polymer composition: 52 mol % D,L-Lactide and 48 mol % Glycolide; inherent viscosity: 0.41 dl/g, 25° C., 0.1% in CHCl₃; grey—RG502H; PLGA; polymer composition: 51 mol % D,L-Lactide and 49 mol % Glycolide; inherent viscosity: 0.19 dl/g, 25° C., 0.1% in CHCl₃; from Boehringer, Ingelheim), whereas the porosity is the absolute alteration in porosity calculated according to example 8. Dependent on the temper temperature used the porosity of the composite material can be adjusted. In this example the highest porosity was achieved at 57° C.

FIG. 6 presents the compression strength in MPa of a composite material derived from β-TCP powder dependent on the temper temperature for two different polymers: (dark) RG502H (PLGA; polymer composition: 51 mol % D,L-Lactide and 49 mol % Glycolide; inherent viscosity: 0.19 dl/g, 25° C., 0.1% in CHCl₃; from Boehringer, Ingelheim) compared to (white) RG503H (PLGA; polymer composition: 52 mol % D,L-Lactide and 48 mol % Glycolide; inherent viscosity: 0.41 dl/g, 25° C., 0.1% in CHCl₃ from Boehringer, Ingelheim) of a β TCP powder/PLGA composite with a polymer to β-TCP ratio of 1, 5:1 (w/w) analogous to example 4 but with a thermal treatment carried out within an oven at different temperatures. The horizontal line represents the maximum compression strength of an isolated vertebral body (X) and the average compression strength of an isolated vertebral body (Y) derived from the literature (Wintermantel et al. 2002). The mechanical properties were measured according to example 9. Dependent on the temper temperature used the compressive strength of the composite material can be adjusted. In this example the highest compressive strength in this example was achieved at 75° C. Surprisingly the inventors found, that the compressive strength was further increased by using a polymer with a lower molecular weight (shorter chain length, lower viscosity) compared to a higher molecular weight (longer chain length, higher viscosity) (e.g. RG502H vs RG503H). By varying the temper temperature in combination with the selected polymer chain length the compressive strength can be fine tuned to establish a composite material where the mechanical stability is improved but nevertheless the porosity is conserved for cell ingrowth into the material for new bone formation. Such fine tuning is matter of routine measures for the skilled person.

FIG. 7 shows the Young's Modulus (E-module) of a composite material derived from β-TCP powder/RG502H composite dependent on the PLGA-β-TCP ratio 1:1 (w/w) (A), 1:1.5 (w/w) (B), 1:3 (w/w) (C) manufactured analogous to example 4 by only varying the PLGA-β-TCP ratio. The mechanical properties were measured according to example 9.

FIG. 8 shows the Young's Modulus (E-module) of a composite material dependent on the β-TCP powder content in percent (%) and various β-TCP powder granule mixtures whereas the total amount of the inorganic phase is constant. The composite material was derived from β-TCP and RG502H in a TCP:polymer ratio of 1, 5:1 (w/w) analogous to example 4. The mechanical properties were measured according to example 9.

FIG. 9 shows the compressive strength from composite materials in MPa derived from β-TCP with RG502H with and without an outer dense structure (e.g., cage) according to the examples 2, 3, 4 and 5. X represents the maximum compressive strength of an isolated vertebral body, Y the average compressive strength of an isolated vertebral body. The mechanical properties were measured according to example 9.

A: The β-TCP powder derived composite material according to example 4

B: The composite material derived from β-TCP granules according to example 2

C: The β-TCP powder derived composite material according to example 5

D: The β-TCP powder derived composite material according to example 3

E: The β-TCP powder derived composite material according to example 4 with an additional thermal compression step using a hot press at approx. 80° C. for 1 min with a compression force of approx. 50 N.

FIG. 10 shows the calculated corresponding Young's Modulus (E-module) in MPa of the composite materials of FIG. 9 A to E. The mechanical properties were measured according to example 9.

FIG. 11 shows the compressive strength in MPa of different composite materials (A to K) derived from β-TCP powder and RG502H (PLGA; polymer composition: 51 mol % D,L-Lactide and 49 mol % Glycolide; inherent viscosity: 0.19 dl/g, 25° C., 0.1% in CHCl₃; from Boehringer, Ingelheim) with a β-TCP polymer ratio of 1, 5:1 according to example 4 with additional fibre reinforcement as described in example 6. X represents the maximum compressive strength of an isolated vertebral body, Y the average compressive strength of an isolated vertebral body derived from the literature (Wintermantel et al., 2002). The mechanical properties were measured according to example 9. The compressive strength (MPa) is shown in brackets.

A: The β-TCP powder derived composite material according to example 4 (3.91)

B: The β-TCP powder derived composite material according to example 4 with an additional thermal treatment at 80° C. for approx. 1 hour (30.02)

C: Composite material according to example 6 with glass fibers (4.17)

D: Composite material according to example 6 with glass fibers with an additional thermal treatment at 80° C. for approx. 1 hour (7.82)

E: Composite material according to example 6 with PGA-fleece (supplied by Synthecon) (5.86)

F: Composite material according to example 6 with PGA-fleece (supplied by Synthecon) with an additional thermal treatment at 80° C. for approx. 1 hour (5.91)

G: Composite material according to example 6 with Nylon scaffold (6.61)

H: Composite material according to example 6 with Nylon scaffold with an additional thermal treatment at 80° C. for approx. 1 hour (15.05)

I: Composite material according to example 6 with Ethisorb (supplied by Ethicon) (8.35)

J: Composite material according to example 6 with Ethisorb (supplied by Ethicon) with an additional thermal treatment at 80° C. for approx. 1 hour (5.1)

K: β-TCP block provided by Curasan (5.1)

FIG. 12 shows pictures of fiber reinforced composite material derived from β-TCP powder and RG502H (PLGA:β-TCP 1:1.5 w/w) according to example 6 after measurement of compressive strength according to example 9.

A: Composite material according to example 6 with glass fibres.

B: Composite material according to example 6 with Nylon scaffold.

C: Composite material according to example 6 with PGA-fleece (supplied by Synthecon)

FIG. 13 shows the stability of pure rhGDF-5 in contact with various organic solvents according to example 10. The graph shows the relative content of unmodified species after contacting pure rhGDF-5 with various organic solvents at room temperature for 1 hour and subsequently drying (grey bar). Afterwards the remaining pure protein was incubated at 60° C. to simulate the conditions at the thermal treatment process (white bar). The solvents used in this Figure were anisole (2), dimethylsulfoxide (DMSO) (3), and glacial acetic acid (4), (1) represents rhGDF-5 as control. The amount of rhGDF-5 was analyzed according to example 17 method A.

FIG. 14 shows the stability of pure parathormone (PTH) in contact with various organic solvents according to example 11. The graph shows the relative content of unmodified species after contacting pure parathormone PTH 1-34 with various organic solvents at room temperature for 1 hour and subsequently drying (grey bar).

Afterwards the remaining pure parathormone PTH 1-34 was incubated at 60° C. to simulate the conditions at the thermal treatment process (white bar). The solvents used were anisole (2), dimethylsulfoxide (DMSO) (3), and glacial acetic acid (4), (1) represents PTH 1-34 as control. The amount of PTH 1-34 was analyzed according to example 17 method B.

FIG. 15 shows the results of solvent screening. The stability of rhGDF-5 coated on β-TCP according to example 13 in contact with various organic solvents after drying at 25° C. without thermal treatment. The graph shows the content of modified species after contacting rhGDF-bound onto beta-TCP with various organic solvents at room temperature for 30 minutes. The white bar represents the amount of rhGDF-5 degradation products (%), the grey bar represents the amount of native rhGDF-5 (relative %) as determined according to example 17 method A. The solvents tested included acetone (2), chloroform (3), ethyl acetate (4), tetrahydrofurane (5), anisole (6), n-butylacetate (7), 1-pentanol (8), dimethylsulfoxide (9), glacial acetic acid (10). (1) represents the stability of rhGDF-5 coated on β-TCP without any solvent treatment.

FIG. 16 shows the stability of rhGDF-5 on β-TCP in contact with various organic solvents and annealing according to example 14. The graph shows the content of modified species after contacting rhGDF-5 bound onto beta-TCP with various organic solvents at room temperature for 30 minutes. After the subsequent drying step the remaining protein coated granules were incubated at 60° C. to simulate the conditions with a thermal treatment step. The white bar represents the amount of rhGDF-5 degradation (%), the grey bar represents the amount of native rhGDF-5(%) as determined according to example 17 method A. The solvents tested included acetone (2), chloroform (3), ethyl acetate (4), tetrahydrofurane (5), anisole (6), n-butylacetate (7), 1-pentanol (8), dimethylsulfoxide (9), glacial acetic acid (10). (1) represents the stability of rhGDF-5 coated on β-TCP without any solvent treatment.

FIG. 17 shows the stability of rhGDF-5 on β-TCP in contact with various organic solvents and annealing after optimized conditions according to example 15 for three well suited solvents as shown in FIGS. 15 and 16. The graph shows the relative content of unmodified species after contacting the rhGDF-5 bound onto beta-TCP with various organic solvents at room temperature for 30 minutes. After the subsequent freeze drying step the remaining protein coated granules were incubated at 60° C. at high vacuum (≦0.1 mbar) to simulate the conditions at thermal treatment process. The solvents used were anisole (2), dimethylsulfoxide (DMSO) (3), and glacial acetic acid (4). (1) represents the stability of rhGDF-5 coated on β-TCP without any solvent treatment.

FIG. 18 shows the stability of rhGDF-5 on β-TCP with various PLGA (RG 502H) shell after optimized lyophilization conditions according to example 18.

The graph shows the relative content of unmodified species after contacting the rhGDF-5 bound onto beta-TCP with a solution of PLGA in DMSO at room temperature for 30 minutes.

After the subsequent freeze drying step the remaining protein coated granules were incubated at 60° C. at high vacuum (≦0.1 mbar) with thermal treatment step to achieve the defined polymeric shell. The bars represent (1) the stability of rhGDF-5 coated on β-TCP without any solvent, (2) rhGDF-5 coated on β-TCP incubated with dimethylsulfoxide (DMSO), (3) rhGDF-5 coated on β-TCP with 4% w/w PLGA, (4) rhGDF-5 coated on β-TCP with 20% w/w PLGA. The amount of rhGDF-5 was analyzed according to example 20.

FIGS. 13 to 18 show that according to the present invention an active agent is conserved and retains its biological activity when encompassed in the composite material of the present invention. The present invention demonstrates that the production steps of the present invention allow for the provision of an intact active agent releasing composite material. The composite material can, thus be prepared to be maintain more than 70% active agent, preferable more than 80% active agent suitable to be retarded released in vivo and allows for a sterile product. The most preferred solvents used within the method of the present invention are those where the amount of native protein is comparable to the control ±5% such as DMSO, glacial acetic acid and anisole.

FIG. 19 represents rhGDF-5 release from coated β-TCP granules with PLGA shell (4% w/w RG 502H in alpha-MEM [minimum essential medium] with 10% FCS at 4° C. without medium exchange) and quantification of residual rhGDF-5 within the granules over the time (in days).

A—Release of rhGDF-5 from the β-TCP granules (determined according to example 25)

B—Residual rhGDF-5 within the β-TCP/PLGA granules (quantification according to example 26).

FIG. 20 represents the release of rhGDF-5 from coated β-TCP granules with PLGA shell according to example 18 (4% w/w and 20% w/w RG 502H) vs rhGDF-5 coated β-TCP granules according to example 12 (without PLGA) in alpha-MEM with 10% FCS at 4° C. without medium exchange. The quantification of rhGDF-5 was done according to example 25.

A—Release of rhGDF-5 from the β-TCP granules (without PLGA shell)

B—Release of rhGDF-5 from coated β-TCP granules with PLGA shell (4% w/w)

C—Release of rhGDF-5 from coated β-TCP granules with PLGA shell (20% w/w)

FIG. 21 represents the release of rhGDF-5 from a composite material derived from β-TCP powder with different polymer solutions and different polymers (in alpha-MEM/10% FKS at 4° C. without medium exchange) over time (in days). The samples were manufactured according to example 22 and the quantification of rhGDF-5 was done according to example 25.

A—Release of rhGDF-5 from the β-TCP granules (without PLGA shell)

B—15% RG502H, β-TCP/polymer ratio of 6.0:1.0

C—30% RG502H β-TCP/polymer ratio of 3.0:1.0

D—30% RG503H β-TCP/polymer ratio of 3.0:1.0

FIG. 19 to 21 show that according to the present invention a sustained release of the active agent as shown for free flowing granules (FIG. 19, 20) as well as the composite 3-dimensional scaffold (FIG. 21) avoiding a high initial burst upon using such a composite material for bone augmentation.

FIG. 22 shows the homogeneity of protein coating according to example 27 step 2. A, represents β-TCP granules without protein coating (rhGDF-5) as a control. B represents the homogenous rhGDF-5 coated granules prepared according to example 12. C shows a similar homogenous rhGDF-5 coating on the granules manufactured according to example 18 compared to B after extraction of the PLGA shell according to example 27 Step 1. D shows PLGA coated granules (without rhGDF-5) according to example 1 after extraction of the PLGA shell according to example 27 step 1 as a control that residual polymer simply do not lead also to a blue staining.

The experiment shows that according to the present invention an active agent such as GDF is homogenously coated on the water insoluble carrier such as β-TCP, also in the presence of water insoluble polymer allowing for retarded release. Removal of the polymer shell, does not affect the homogeneous coating of active agent and, thus, allows for a combination of retarded release and optimized active agent effect. This result applies to method A and method B of the present invention.

### Table 1: Freeze-Drying Parameters for Protein or Peptide Coated β-TCP

This table shows the details of the lyophilization program for the manufacturing of protein or peptide onto β-TCP according to example 12.

Table 2: Freeze-Drying Parameters for the Manufacturing of Protein or Peptide Loaded PLGA/β-TCP Composite

This table shows the details of the lyophilization program for the manufacturing of protein or peptide loaded composite PLGA/β-TCP granules and composite materials to achieve a minimum solvent induced protein- or peptide degradation.

TABLE 1
Freeze-drying parameters for protein or peptide coated β-TCP

| | | Shelf | | | |
|---|---|---|---|---|---|
| | | temper- | Time per | | |
| | | ature | Step | Total time | Pressure |
| | | [° C.] | [hh:mm] | [hh:mm] | [mbar] |
| | Start precooled | −20 | 00:00 | 00:00 | 1000 |
| | shelves | | | | |
| 1 | Loading | −20 | 01:00 | 01:00 | 1000 |
| 2 | Freezing (ramp) | −20 | 01:20 | 02:20 | 1000 |
| 3 | Freezing I | −20 | 00:30 | 02:50 | 1000 |
| 4 | Incubation I | −5 | 00:30 | 03:20 | 1000 |
| 5 | Incubation II | −5 | 01:30 | 04:50 | 1000 |
| 6 | Freezing (ramp) | −20 | 00:30 | 05:20 | 1000 |
| 7 | Freezing II | −20 | 01:00 | 06:20 | 1000 |
| 8 | — | −20 | 00:00 | 06:20 | 1000 |
| 9 | — | −20 | 00:00 | 06:20 | 1000 |
| 10 | Ice condenser | −20 | 00:30 | 06:50 | 1000 |
| | precooling | | | | |
| 11 | Adjust vacuum | −20 | 00:30 | 07:20 | 0.2 |
| 12 | Primary drying (ramp) | 25 | 02:30 | 09:50 | 0.2 |
| 13 | Primary drying I | 25 | 10:00 | 19:50 | 0.2 |
| 14 | Secondary drying I | 25 | 00:00 | 19:50 | 0.2 |
| | (ramp) | | | | |
| 15 | Secondary drying I | 25 | 00:00 | 19:50 | 0.2 |
| 16 | Secondary drying II | 25 | 00:00 | 19:50 | 0.2 |
| | (ramp) | | | | |
| 17 | Secondary drying II | 25 | 00:00 | 19:50 | 0.2 |
| 18 | Secondary drying III | 25 | 00:00 | 19:50 | 0.2 |
| | (ramp) | | | | |
| 19 | Secondary drying III | 25 | 00:00 | 19:50 | 0.2 |
| 20 | Venting with sterile N₂ | 25 | 00:10 | 20:00 | 800 |

TABLE 2
Freeze-drying parameters for the manufacturing of
protein or peptide loaded PLGA/β-TCP composite

| | | Shelf | | | |
|---|---|---|---|---|---|
| | | temper- | Time per | | |
| | | ature | Step | Total time | Pressure |
| | | [° C.] | [hh:mm] | [hh:mm] | [mbar] |
| | Start precooled | −5 | 00:10 | 00:10 | 1000 |
| | shelves | | | | |
| 1 | Loading | −5 | 00:15 | 00:25 | 1000 |
| 2 | Freezing (ramp) | −45 | 00:30 | 00:55 | 1000 |
| 3 | Freezing I | −45 | 01:30 | 02:25 | 1000 |
| 4 | Incubation I | −45 | 00:00 | 02:25 | 1000 |
| 5 | Incubation II | −45 | 00:00 | 02:25 | 1000 |
| 6 | Freezing (ramp) | −45 | 00:00 | 02:25 | 1000 |
| 7 | Freezing II | −45 | 00:00 | 02:25 | 1000 |
| 8 | — | −45 | 00:00 | 02:25 | 1000 |
| 9 | — | −45 | 00:00 | 02:25 | 1000 |
| 10 | Ice condenser | −45 | 00:30 | 02:55 | 1000 |
| | precooling | | | | |
| 11 | Adjust vacuum | −45 | 00:30 | 03:25 | 0.056 |
| 12 | Primary drying (ramp) | 10 | 04:00 | 07:25 | 0.056 |
| 13 | Primary drying I | 10 | 23:00 | 30:25 | 0.056 |
| 14 | Secondary drying I | 15 | 04:00 | 34:25 | 0.056 |
| | (ramp) | | | | |
| 15 | Secondary drying I | 15 | 20:00 | 54:25 | 0.056 |
| 16 | Secondary drying II | 20 | 02:00 | 56:25 | 0.056 |
| | (ramp) | | | | |
| 17 | Secondary drying II | 20 | 04:00 | 60:25 | 0.056 |
| 18 | Secondary drying III | 60 | 00:30 | 60:55 | 0.056 |
| | (ramp) | | | | |
| 19 | Secondary drying III | 60 | 00:50 | 61:15 | 0.056 |
| 20 | Stand-by | 20 | 01:00 | 62:15 | 0.056 |
| 21 | Venting with sterile N₂ | 20 | 00:10 | 62:25 | 800 |

### DETAILED DESCRIPTION OF THE PREFERRED EMBODIMENTS

For the purpose of promoting an understanding of the principles of the invention, references will be made to certain embodiments thereof and specific language to be used to describe the same. It will nevertheless be understood that no limitation of the scope of the invention is thereby intended, such alterations, further applications and modifications of the principle of the invention as illustrated herein being contemplated as would normally occur to one skilled in the art to which the invention relates.

The term “free flowing granules” as used in accordance to the present invention refers to granulate ceramic made of for example beta-tricalcium phosphate. The grains of the granulate ceramic can vary dependent on the indication and use of the material, for example the grain size varies between 50 and 150 μm for smaller bone defects, 150 to 500 μm for larger bone defects, 500 to 1000 μm, up to 5000 μm or larger or a mixture thereof. Free flowing means that the material is easily separable for example by shaking or slight pressure from the packaging material and that the granules can be easily adapted to the defect site.

The term “composite material” as used in accordance to the present invention refers to an entity, which comprises at least three components as set forth below. In one embodiment the material is a drug delivery system with porous scaffold. In a further embodiment the material are free flowing granules used as a drug delivery system with sustained release. These materials are preferably suitable for surgical defect filling and tissue regeneration. In another embodiment the material is a drug delivery system for the controlled release of active substances after implantation. The material of the present invention may consist of any device suitable for implantation, including a prosthetic device, sponge, cage or ceramic block, preferably of free flowing granules, composite 3-dimensional scaffolds with macroporosity and/or microporosity. Preferably such composite device is not a microsphere polymer composite comprising of polymer microspheres or ceramic containing microsphere derived composites such as described in U.S. Pat. No. 5,766,618, by Khan et al., 2004 and Ruhe et al., 2004. In these polymer microspheres or ceramic containing microsphere derived composites there is a process related loss of protein due to the limited inclusion efficacy of the protein within the microspheres. Furthermore, the combination of microspheres and a calcium phosphate cement resembles the disadvantages of a pure calcium phosphate cement system (e.g., no macroporosity, addition of a solution shortly before application) as described above. The high temperature process combined with an unfavourable solvent inhibits the combination with an active agent.

Preferably the composite material such as composite 3-dimensional scaffolds with macroporosity has a porosity of less than 80%, more preferably less than 70% or most preferably a porosity below 65%. The porosity can be adjusted with for example the temperature of the thermal treatment dependent on the compatibility with the active agent (see FIG. 5), the particle size of the water insoluble inorganic filler and/or the amount of the water insoluble polymer. A higher porosity of a macroporous three-dimensional composite would be unfavourable due to the reduced mechanical stability of the material as its shown for conventional composite materials with higher porosity.

The term composite material and composite device are used interchangeable. The term composite 3-dimensional scaffold, composite 3 dimensional material or solid three dimensional scaffold or material are used synonymous.

The term “microporosity” means a porosity with pores of about 10 μm diameter or smaller. Preferably theses micropores are interconnecting with other micropores or insulated macropores forming a network of channels.

The term “macroporosity” means a porosity with pore size of equal or more than 10, preferably more than 25, most preferably more than 100 μm diameter and more sufficient for ingrowth of living cell to support new bone formation with the composition. Preferably said macroporous scaffold has macropores throughout the composite material. More preferably said macroporous three-dimensional scaffold has interconnecting macropores establishing a network of channels in which progenitor cells and bone cells can migrate.

The term “interconnecting pores” means a network of pores and pore channels with micro- and macropores throughout the material most preferably macropores and macrochannels creating a porosity with a pore size sufficient for cell infiltration such as bone cells or precursor cells. Preferably the pore size of the interconnecting pores have a diameter of equal or more than 100 μm.

One of the components of said material is a water insoluble solid filler, the so-called “carrier” or “inorganic matrix”. Preferably, water insoluble solid filler consists of ceramics. Preferably, said carrier is a calcium phosphate. Most preferably said inorganic matrix is a calcium phosphate, which is beta-tricalcium phosphate, alpha-tricalcium phosphate, apatite or a calcium phosphate containing cement. Alternatively, said carrier is selected from the group consisting of calcium carbonate, magnesium carbonate, magnesium oxide, magnesium hydroxide or silicium dioxide based materials (e.g., bioglass). Preferably the carrier is bioresorbable. Most preferably, the water insoluble solid filler is a high soluble calcium phosphate, preferably a tricalcium phosphate, since these fillers are bioresorbable whereas sintered highly crystalline hydroxyapatite are less or non bioresorbable.

Said ceramics may have a particularly high surface due to the small particles size or the presence of macro- and micropores. In a preferred embodiment, said macropores have a diameter of 100 to 400 μm. In another preferred embodiment, said micropores have a diameter of less than 10 μm. In still another preferred embodiment, the pores are interconnected to allow the influx of coating substances in the material preparation as well as in-growth of bone and tissue cells in the application in vivo.

The term “carrier” encompasses three-dimensional matrices, such as the above-mentioned ceramics and ceramic/polymer composites. The carrier, preferably, has an enlarged surface due to the small particles size or the formation of macro- and micropores during the manufacturing process.

The carrier comprised by the material of the invention may be brought into a suitable form for administration of the material in vivo, such as solid composite materials in form of blocks, cubes, discs or granules. In addition, the composite carrier may be coated onto a metallic surface.

In a preferred embodiment, the carrier containing calcium phosphate is in a granular form, more preferably in the form of free flowing granules. Granular products as moldable systems are well established for surgical defect filling especially in orthopedic indications (Draenert et al., 2001). Therefore it is important to meet this preferred application form. In an alternatively preferred embodiment this granular form is used as a starting material for forming a solid three dimensional scaffold with micro- and macroporosity, preferably with a high mechanical strength to be used not only for non-load bearing applications. This solid three dimensional scaffold is preferably formed by annealing the PLGA coated granules. In another preferred embodiment, the carrier containing calcium phosphate is in a powder form as a starting material for forming a solid three dimensional scaffold preferably with the manifestation of a load bearing three-dimensional implant with mechanical properties preferably similar to trabecular bone.

The term “calcium phosphate” encompasses compositions comprising calcium ions (Ca²⁺), phosphate ions (PO₃³⁻), optionally, further ions like hydroxyl ions (OH⁻), carbonate (CO₃²⁻) or magnesium (Mg²⁺) or other ions which are suitable for the carrier of the present invention. The calcium phosphates as used in accordance with the present invention are crystals having a three dimensional structure suitable for the material of the present invention as set forth above. Said calcium phosphates are particularly well suited as carriers for the material of the present invention. Their in vivo properties have been described in Hotz, 1994, Gao, 1996, and in WO98/21972. A list of preferred and well-known calcium phosphates is given above.

The second component of the material of the present invention is a water insoluble polymer. Preferably said polymer is a “biocompatible”, a “biodegradable” or a “bioresorbable” polymer.

The term “biocompatible” means the ability of a material to perform with an appropriate host response in a specific application (Wintermantel et al., 2002). Furthermore the term “biocompatible” means, that the material does not exhibit any toxic properties and that it does not induce any immunological or inflammatory reactions after application.

The term “biodegradable” specifies materials for example polymers, which break down due to macromolecular degradation with dispersion in vivo but for which no proof exists for the elimination from the body. The decrease in mass of the biodegradable material within the body is the result of a passive process, which is catalysed by the physicochemical conditions (e.g. humidity, pH value) within the host tissue.

The term “bioresorbable” specifies materials such as polymeric materials, which underwent degradation and further resorption in vivo; i.e. polymers, which are eliminated through natural pathways either because of simple filtration of degradation by-products or after their metabolization. Bioresorption is thus a concept, which reflects total elimination of the initial foreign material. In a preferred embodiment of the present invention said bioresorbable polymer is a polymer that undergoes a chain cleavage due to macromolecular degradation in an aqueous environment. It has to be mentioned that the term “resorption” always describes an active process.

In a preferred embodiment of the material or the method of the invention said bioresorbable polymer is a polymer that undergoes a chain cleavage due to macromolecular degradation in an aqueous environment.

More preferably said water insoluble polymer is an aliphatic polymer preferably with a glass transition temperature above 35° C. of the pure substance and an inherent viscosity of 0.1 to 0.4 dl/g, preferably 0.1 to 0.3 dl/g, wherein the inherent viscosity is determined at 25° C. and 0.1% solution in chloroform.

Alternatively, said polymer is selected from the group consisting of polyethylene (PE), polypropylene (PP), polyethylenerephthalate (PET), polyglactine, polyamide (PA), polymethylmethacrylate (PMMA), polyhydroxymethylmethacrylate (PHEMA), polyvinylchloride (PVC), polyvinylalcohole (PVA), polytetrafluorethylene (PTFE), polyetheretherketone (PEEK), polysulfon (PSU), polyvinylpyrolidone, polyurethane or polysiloxane. These polymers are at least biocompatible.

More preferably, said polymer is selected from the group consisting of poly(epsilon-hydroxy acids), poly(ortho esters), poly(anhydrides), poly(aminoacids), polyglycolid (PGA), polylactid (PLLA), poly(D,L-lactide) (PDLLA), poly(D,L-lactide co-glycolide) (PLGA), poly(3-hydroxybutyricacid) (P(3-HB)), poly(3-hydroxy valeric acid) (P(3-HV)), poly(p-dioxanone) (PDS), poly(epsilon-caprolactone) (PCL), polyanhydride (PA), copolymers (e.g., diblock copolymers PLGA-PEG), terpolymers, blockcopolymers, combinations, mixtures thereof. These polymers are biocompatible and bioresorbable.

Even more preferably, said polymer is an amorphous polymer, most preferably PLGA with a glycolic acid composition between 25 to 70 mol % (m %) glycolic acid, preferable 50 m % within the polymer chain). If the di-lactic acid is used, the amorphous region extends from 0-70 m % glycolic acid within the polymer chain. Polymers with this glycolic acid composition are totally amorphous and therefore exhibit only a glass transition and do not crystallize. If the polymer is heated above this glass transition temperature these polymers become viscous, a prerequisite to achieve a homogeneous coating of the polymer onto the ceramic carrier. Polymers outside the above mentioned amorphous range do not show this specific behavior and thus not be capable. Additional by changing the lactic acid:glycolic acid ratio, it is possible to tailor the rate of degradation to that required for the specific application or use. PLGA (50:50) means a lactic acid:glycolic acid monomer ratio in the polymer chain of 1:1.

The third component of the material of the present invention is an “active agent”. The term “active agent” comprises a polypeptide or a small molecule drug which is immobilized on and/or in the carrier or dispersed within the polymer. Preferably, said polypeptide or drug is homogeneously distributed on the calcium phosphate containing carrier and/or homogenously dispersed within the polymer.

The term “osteoconductive” refers to substrates that provide a favourable scaffolding for vascular ingress, cellular infiltration and attachment, cartilage formation, and calcified tissue deposition. Osteoconductive materials may support osseous generation via the scaffolding effect (Kenley, R. A., 1993).

The term “osteoinductive” refers to the capability of the transformation of mesenchymal stem cells into osteoblasts and chondrocytes. A prerequisite for osteoinduction is a signal which is distributed by the material into the surrounding tissues where the aforementioned osteoblast precursors become activated. Osteoinduction as used herein encompasses the differentiation of mesenchymal cells into the bone precursor cells, the osteoblasts. Moreover, osteoinduction also comprises the differentiation of said osteoblasts into osteocytes, the mature cells of the bone. Moreover, also encompassed by osteoinduction is the differentiation of mesenchymal cells into chondrocytes. In particular in the long bones, the chondroblasts and the chondrocytes residing in the perichondrium of the bone can also differentiate into osteocytes. Thus, osteoinduction requires differentiation of undifferentiated or less-differentiated cells into osteocytes which are capable of forming the bone. Thus, a prerequisite for osteoinduction is a signal which is distributed by the material into the surrounding tissues where the aforementioned osteocyte precursors usually reside. As has been described above, the osteoinductive proteins or peptides used in accordance with the present invention are sustained released from the material after implantation and are distributed efficiently in the surrounding tissues. Moreover, the proteins and peptides encompassed by the present invention have osteoinductive properties in vivo. For example, it is well known in the art that the Transforming Growth Factor-β (TGF-β) superfamily encompasses members which have osteoinductive properties. Individual members of said TGF-β superfamily which have particular well osteoinductive properties are listed infra. In conclusion, the osteoinductive proteins or peptides of the material of the present invention after having been released from the carrier serving as a osteoinductive signal for the osteocyte precursors of the tissue surrounding the side of implantation of the material.

The term “osteogenic” describes the synthesis of new bone by osteoblasts. In accordance with the present invention, preexisting bone in the surrounding of the side of implantation of the material grows into the material using the structure of the material as a matrix onto which the osteocytes can adhere.

The term “osteoinductive polypeptide” refers to polypeptides, such as the members of the Transforming Growth Factor-β (TGF-β) superfamily, which have osteoinductive properties.

In a further preferred embodiment of the material or the method of the invention said osteoinductive protein is a member of the TGF-β family.

The TGF-β family of growth and differentiation factors has been shown to be involved in numerous biological processes comprising bone formation. All members of said family are secreted polypeptides comprising a characteristic domain structure. On the very N-terminus, the TGF-β family members comprise a signal peptide or secretion leader. This sequence is followed at the C-terminus by the prodomain and by the sequence of the mature polypeptide. The sequence of the mature polypeptide comprises seven conserved cysteins, six of which are required for the formation of intramolecular disulfide bonds whereas one is required for dimerization of two polypeptides. The biologically active TGF-β family member is a dimer, preferably composed of two mature polypeptides. The TGF-β family members are usually secreted as proteins comprising in addition to the mature sequence the prodomain. The prodomains are extracellularly cleaved off and are not part of the signalling molecule. It has been reported, however, that the prodomain(s) may be required for extracellular stabilization of the mature polypeptides.

In the context of the present invention, the term “TGF-β family member” or the proteins of said family referred to below encompass all biologically active variants of the said proteins or members and all variants as well as their inactive precursors. Thus, proteins comprising merely the mature sequence as well as proteins comprising the mature protein and the prodomain or the mature protein, the prodomain and the leader sequence are within the scope of the invention as well as biologically active fragments thereof. Whether a fragment of a TGF-β member has the biological activity can be easily determined by biological assays described, e.g. in: Katagiri et al., 1990; Nishitoh et al., 1996.

Preferably, the biological activity according to the invention can be determined by in vivo models as described in the accompanied Examples. Such assays for determination of the activity include alkaline phosphatase (ALP) assay well known to the expert in the field. Furthermore, encompassed by the present invention are variants of the TGF-β members which have an amino acid sequences being at least 75%, at least 80%, at least 90%, at least 95%, at least 96%, at least 97%, at least 98% or at least 99% identical to the amino acid sequences of the members of the TGF-β family.

An overview of the members of the TGF-β superfamily is given in: Wozney J M, Rosen V (1998): Bone morphogenetic protein and bone morphogenetic protein gene family in bone formation and repair. Clin Orthop 346: 26-37. The amino acid sequences of the members of the TGF-β family can be obtained from the well known databases such as Swiss-Prot via the internet (http://www.expasy.ch/sprot/sprot-top.html). Amino acid sequences for BMP-2, BMP-7 and GDF-5, members of the TGF-family with a particularly high osteoinductive potential, are also shown in SEQ ID No: 1 to 3, respectively. Amino acid sequences for BMP-2, BMP-7 and GDF-5, members of the TGF-β family with a particularly high osteogenic potential, are also shown in SEQ ID No: 1 to 3, respectively.

More preferably, said member of the TGF-β family is a member of the BMP subfamily. The members of the Bone Morphogenetic Protein (BMP) subfamily have been shown to be involved, inter alia, in the induction and re-modeling of bone tissue. BMPs were originally isolated from bone matrix. These proteins are characterized by their ability to induce new bone formation at ectopic sites. Various in vivo studies demonstrated the promotion of osteogenesis and chondrogenesis of precursor cells by BMPs and raise the possibility that each BMP molecule has distinct role during the skeletal development. More details about the molecular and biological properties of the BMPs are described in: Wozney J M, Rosen V (1998): Bone morphogenetic protein and bone morphogenetic protein gene family in bone formation and repair. Clin Orthop 346: 26-27, Schmitt J, Hwang K, Winn, S R, Hollinger J (1999): Bone morphogenetic proteins: an update on basic biology and clinical relevance. J Orthop Res 17: 269-278 and Lind M (1996): Growth factors: possible new clinical tools. A review. Acta Orthop Scand 67: 407-17.

The osteoinductive polypeptide of the present invention is preferably selected from the group consisting of BMP-1, BMP-2, BMP-3, BMP-4, BMP-5, BMP-6, BMP-7, BMP-8, BMP-9, BMP-10, BMP-11, BMP-12, BMP-13, BMP-14, BMP-15 and BMP-16. Most preferably, said member of the BMP family is BMP-2 or BMP-7.

The amino acid sequence for the preproform of BMP-2 is deposited under Swiss-Prot Accession number P12643 and is shown below. Amino acids 1 to 23 correspond to the signal sequence, amino acids 24 to 282 correspond to the propeptide and amino acids 283 to 396 correspond to the mature protein. The amino acid sequence for the preproform of BMP-7 is deposited under Swiss-Prot Accession number P18075 or shown in SEQ ID No: 2. Amino acids 1 to 29 correspond to the leader sequence, amino acids 30 to 292 correspond to the proform and amino acids 293 to 431 correspond to the mature protein. Preferably, BMP-2 or BMP-7 refers to the preproform, to the proform or to the mature BMP-2 or BMP-7 peptide, respectively. Moreover also encompassed are fragments of said proteins having essentially the same biological activity, preferably osteoinductive properties. More sequence information for BMP-2 and BMP-7 is provided below.

Alternatively, the osteoinductive polypeptide of the present invention is selected from another TGF-β family, i.e. the GDF family.

Growth and Differentiation Factor (GDF) have been also shown to be involved, inter alia, in the induction and re-modeling of bone tissue. Growth Differentiation Factor 5 (GDF-5), also known as cartilage-derived morphogenetic protein 1 (CDMP-1) is a member of subgroup of the BMP family, which also includes other related proteins, preferably, GDF-6 and GDF-7. The mature form of the protein is a 27 kDa homodimer. Various in vivo and in vitro studies demonstrate the role of GDP-5 during the formation of different morphological features in the mammalian skeleton. Mutations of GDF-5 are responsible for skeletal abnormalities including decrease of the length of long bones of limbs, abnormal joint development in the limb and sternum (Storm & Kingsley (1999), Development Biology, 209, 11-27). The amino acid sequence between mouse and human is highly conserved.

Preferably, the osteoinductive polypeptide of the present invention is selected from the group consisting of GDF-1, GDF-2, GDF-3, GDF-4, GDF-5, GDF-6, GDF-7, GDF-8, GDF-9, GDF-10 and GDF-11. Most preferably, said member of the GDF subfamily is GDF-5.

The amino acid sequence for the preproform of GDF-5 is deposited under Swiss-Prot Accession number P 43026 or shown in SEQ ID No: 3. Amino acids 1 to 27 correspond to the leader sequence, amino acids 28 to 381 correspond to the proform and amino acids 382 to 501 correspond to the mature protein. Preferably, GDF-5 refers to the preproform, to the proform or to the mature GDF-5 peptide. Moreover also encompassed are fragments of GDF-5 having essentially the same biological activity, preferably osteoinductive properties. Most preferably, said fragment comprises amino acids 383 to 501 of the sequence shown in SEQ ID No: 3.

The following tables show amino acid sequences for BMP-2, BMP-7 and GDF-5:

Human BMP-2 (Swiss-Prot Prim. Accession Number P12643); SEQ ID No. 1:

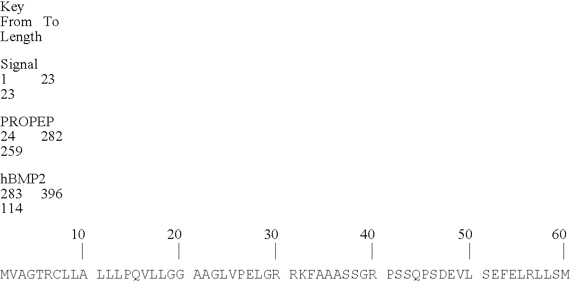

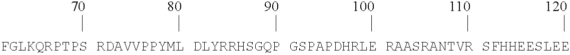

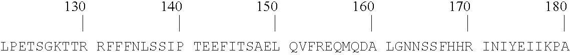

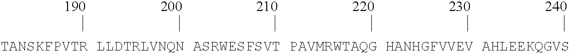

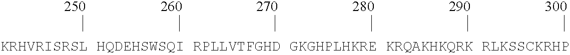

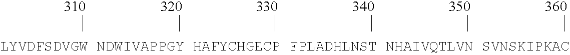

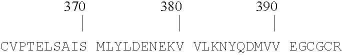

References
[1]
SEQUENCE FROM NUCLEIC ACID.
MEDLINE = 89072730; PubMed = 3201241;
Wozney J. M., Rosen V., Celeste A. J., Mitsock L. M., Whitters M. J., Kriz R. W., Hewick R. M., Wang E. A.;
“Novel regulators of bone formation: molecular clones and activities.”;
Science 242:1528-1534 (1988).
[2]
X-RAY CRYSTALLOGRAPHY (2.7 ANGSTROMS) OF 292-396.
MEDLINE = 99175323; PubMed = 10074410;
Scheufler C., Sebald W., Huelsmeyer M.;
“Crystal structure of human bone morphogenetic protein-2 at 2.7 A resolution.”;
J. Mol. Biol. 287:103-115 (1999).

Human BMP-7 (Swiss-prot Prim. Accession. Number: P18075); SEQ ID No. 2:

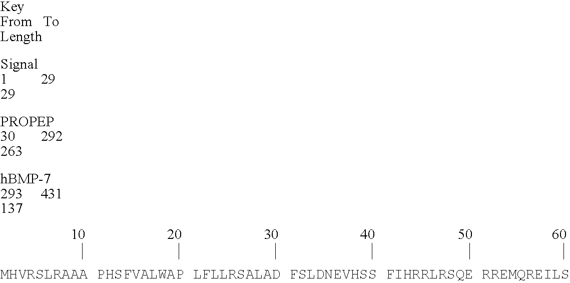

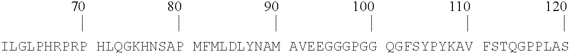

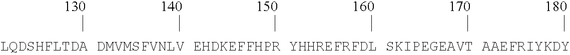

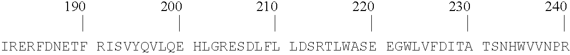

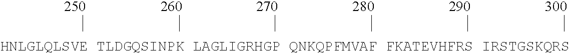

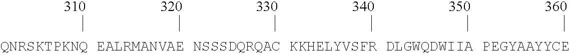

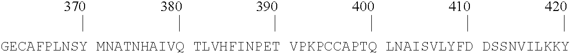

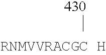

References
[1]
SEQUENCE FROM NUCLEIC ACID, AND PARTIAL SEQUENCE.
TISSUE = Placenta;
MEDLINE = 90291971; PubMed = 2357959;
Oezkaynak E., Rueger D. C., Drier E. A., Corbett C., Ridge R. J., Sampath T. K., Oppermann H.;
“OP-1 cDNA encodes an osteogenic protein in the TGF-beta family.”;
EMBO J. 9:2085-2093 (1990).
[2]
SEQUENCE FROM NUCLEIC ACID.
MEDLINE = 91088608; PubMed = 2263636;
Celeste A. J., Iannazzi J. A., Taylor R. C., Hewick R. M., Rosen V., Wang E. A., Wozney J. M.;
“Identification of transforming growth factor beta family members present in bone-inductive protein purified from bovine bone.”;
Proc. Natl. Acad. Sci. U.S.A. 87:9843-9847 (1990).
[3]
X-RAY CRYSTALLOGRAPHY (2.8 ANGSTROMS) OF 293-431.
MEDLINE = 96149402; PubMed = 8570652;
Griffith D. L., Keck P. C., Sampath T. K., Rueger D. C., Carlson W. D.;
“Three-dimensional structure of recombinant human osteogenic protein 1: structural paradigm for the transforming growth factor beta superfamily.”;
Proc. Natl. Acad. Sci. U.S.A. 93:878-883 (1996).

Human GDF-5 (Swiss-Prot Prim. Accession Number: P 43026); SEQ ID No. 3:

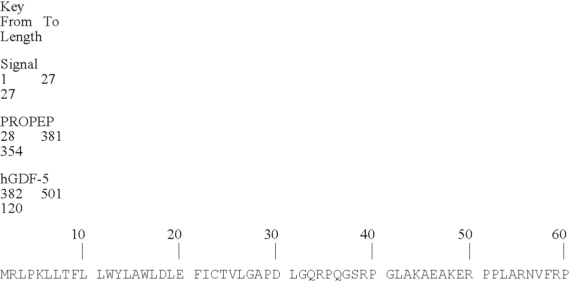

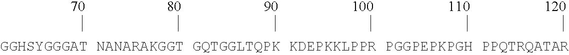

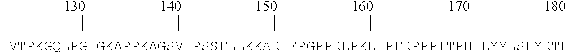

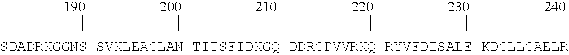

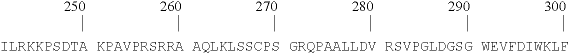

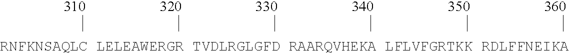

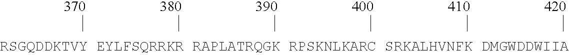

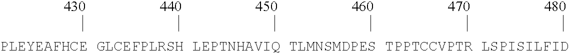

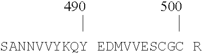

References
[1]
SEQUENCE FROM NUCLEIC ACID.
TISSUE = Placenta;
MEDLINE = 95071375; PubMed = 7980526;
Hoetten G., Neidhardt H., Jacobowsky B., Pohi J.;
“Cloning and expression of recombinant human growth/differentiation factor 5.”;
Biochem. Biophys. Res. Commun. 204:646-652 (1994).
[2]
SEQUENCE FROM NUCLEIC ACID.
TISSUE = Articular cartilage;
MEDLINE = 95050604; PubMed = 7961761;
Chang S., Hoang B., Thomas J. T., Vukicevic S., Luyten F. P., Ryba N. J. P., Kozak C. A., Reddi A. H., Moos M.;
“Cartilage derived morphogenetic proteins. New members of the transforming growth factor-beta superfamily predominantly expressed in long bones during human embryonic development.”;
J. Biol. Chem. 269:28227-28234 (1994).

Also encompassed by the present invention are embodiments, wherein said active agent is selected from hormones, cytokines, growth factors, antibiotics and other natural and/or synthesized drug substances like steroids, prostaglandines etc.

Preferably, said active agent is parathyroid hormone (PTH) and/or PTH 1-34 peptide.

Optionally the carrier is first homogenous coated with an active agent or the active agent is homogenous solved or dispersed within the polymer solution and coated intermingled within the polymer onto the carrier (FIG. 22). In a preferred embodiment the active agent such as rhGDF-5 or other bone morphogenetic proteins like BMP-2 is first homogenous coated onto the β-TCP carrier and surrounded by a shell of polymer.

The term “content of the intact active agent” means that at least 70% of the active agent is stable, more preferably 80%, most preferably 90% over the whole manufacturing process. Further details on how to determine intact active agent are described further below for “compatible with the active agent”. To increase the long time stability of the active agent in the final product the composite material is stored under inert atmosphere (e.g., nitrogen), preferably within the final packaging material.

The material of the present invention may optionally, comprise additional excipients. These excipients serve the stabilization of the protein or peptide, e.g., saccharides, amino acids, polyols, detergents or maintenance of the pH, e.g., buffer substances.

The term “polymer to carrier ratio of the material” means the mass or weight ratio of water insoluble polymer to water insoluble filler of the composite material of the present invention.

The material of the present invention such as the composite 3-dimensional scaffold is macroporous and/or microporous dependent on the carrier educt particle size of the carrier such as beta-tricalcium phosphate used for manufacturing. A microporous scaffold is obtained using a carrier in powder form whereas the macroporous scaffold is obtained using a carrier in granular form, preferably with a particle size of greater than 100 μm, more preferably of about 200 μm or larger, most preferably between 500 and 4000 μm.

The term “particle size” according to the present invention means a distribution of the size diameter of the material such as tricalcium phosphate, microns (μm), which can be determined by laser diffraction. A specific particle size range of material can be for example achieved by sieving.

The term “powder” relates to a solid state with an average particle size of less then 50 μm. The term “educt” means a starting material or intermediate compound such as a water insoluble solid filler material, which is not the final product (composite material).

The term “compressive strength” means the maximum compressive stress the test sample was able to withdraw. Methods for determination of the compressive strength are well known to experts in the field and are further described in example 9 according to EN DIN ISO 604.

The term “Young's modulus” is calculated from the recorded data from the compression test. A synonym for Young's modulus is compressive modulus or E-modulus. Methods for determination of the Young's modulus are well known to experts in the field and are further described in example 9 according to EN ISO DIN 604.

The release of the active agent into the surrounding tissue after implantation can be determined in vitro by various methods such as those described in the examples. Preferably the release is a sustained release with a low initial release of the active agent and further additional release over time. The term sustained release and retarded release can be used synonymous. Preferably the sustained release is at least decreased to 80% compared to the water insoluble polymer-free granules within 2 days as determined in an assay described in example 24, preferably 60% within 2 days, more preferably 50% within 6 days, most preferably 50% within 7 days.

In a preferred embodiment the material of the present invention is free of toxic substances. Preferably such toxic substances are already avoided in the production process, as their production requires additional expenditure due to required removal steps during the production process and necessary expensive means for highly sensitive chemical analysis.

The term “toxic substances”, in particular, encompasses those toxic organic solvents and additives which are used by the methods described in the art, which are classified by the ICH as class 2 solvents (ICH Topic Q 3 C Impurities: Residual Solvents) e.g. methylene chloride. Said substances may cause systemic or local toxic effects, inflammation and/or other reactions after implantation of materials containing said substances. Said prior art materials are therapeutically less acceptable due to said undesirable side effects, which cannot be avoided by the conventionally coating methods described in the art. Moreover, the international guidance for the development of therapeutic proteins require that in the manufacturing process harmful and toxic substances should be avoided (for details see: International Conference on Harmonization (ICH), Topic Q3C; www.emea.eu.int/). However, the material of the present invention or a material, which is obtainable by the method of the present invention is, advantageously, free of said class 1 classified toxic substances. Moreover the present invention contains only solvents classified as class 3 by the ICH Topic Q 3C and, therefore, therapeutically well acceptable and fulfils the requirements of the regulatory authorities. Preferably the same requirements as for solvents in common are valid for the polymer and the water insoluble solid filler of the material of the present invention.

Moreover, in a further preferred embodiment of the material or the method of the invention said material is free of infectious material.

Besides toxic substances, infectious material comprised by the material may cause severe infections in a subject into which the material has been transplanted. Potentially infectious gelatine derived from bovine or procine bones is, however, used as a protecting protein in many state of the art methods (Lind et al., 1996).

Solutions sufficient for the two methods A and B of the present invention (used in step (e) of method A and step (a) of method B, respectively) to produce the material of the present invention preferably have a melting point >−40° C. and a boiling point <200° C.

The term “solution” for dissolving the polymer according to the present invention relates to pharmaceutical acceptable organic solvents capable to dilute the polymer and which are compatible with the active agent.

The term “compatible with the active agent” means that at least 70% of the active agent is stable as determined in the obtained composite material, more preferably 80%, most preferably 90% when analyzed as described in example 10. Stability of the active agent is measured by determination of the degradation, aggregation, oxidation and/or cleavage of the agent according to standard methods such as alteration in mass detection and those described in the examples such as RP-HPLC.

Furthermore, these solvents should be non-toxic in vivo and are pharmaceutically accepted for parental applications at least according to the ICH guidance (ICH Topic Q 3 C Impurities: Residual Solvents). The solvent needs to by dryable under reduced pressure and freeze dryable. Preferably the vapor pressure should be above the vapor pressure of DMSO at ambient temperature, preferable above 0.6 hPa. Solvents that are not useful for the present invention e.g. because of their toxicity include chloroform, acetone, benzole, toluole, methylene chloride, xylole. Solvents, which induce degradation of the active agent for example inactivation of rhGDF-5 such as tetrahydrofurane (THF), are highly undesirable.

Preferable this solution contains a solvent selected from anisole, tetramethylurea, acetic acid, dimethylsulfoxide and tert-butanol (2-methyl-2-propanole trimethylcarbinole-butyl alcohol), acetone, 1-butanole, 2-butanole, butyl acetate, tert-butylmethyl ether, cumene, ethanole, ethyl acetate, dieethylether, ethylformate, formic acid, isobutyl acetate, isopropylacetate, methyl acetate, 3-methyl-1-butanol, methylethyl ketone, methylisobutylketone, 2-methyl-1-propanol, pentane, 1-pentanol, 2-propanol and propylacetate. Most preferred are acetic acid, dimethylsulfoxide and anisole.

The terms “homogeneously distributed” and “homogeneously coated” mean that on average nearly identical amounts of the active agent are present in each and every area of said composite carrier. This area preferably includes the pores of a porous matrix. Homogenous distribution is a prerequisite for efficient release and activity of the active agent into the tissue surrounding the site of implantation. Moreover, it is to be understood that the active agent is not aggregated and partially or entirely inactivated due to precipitation or micro-precipitation, rather attachment of biologically active, non-aggregated proteins is to be achieved by homogenous coating. Said homogenous distribution can be achieved by the two above methods of the present invention.

The homogenous coating of the carrier with said active agent and the simultaneous and/or additional homogeneous coating with the bioresorbable polymer do achieve an onion-like layer structure which acts in two manners as a protective film and as diffusion barrier to slow down the dissolution of the protein or peptide to achieve a sustained release. The described methods A and B allow the homogenous distribution and immobilization of the osteoinductive active agent into and/or on the carrier and the sustained release of the active agent due to the polymeric component.

The efficacy of the coating process is, furthermore, supported by the carrier due to capillary forces resulting from the presence of numerous, preferably interconnected macro- and micro pores which due to their size are capable of soaking the solutions into the pores.

Moreover, in contrast to other methods described in the art, e.g., in WO98/21972, the active agent is—according to the methods A and B of the present invention—applied by attachment to the carriers from the soluble state to achieve a homogeneous coating. The findings underlying the present invention demonstrate that the aggregation of the proteins can be avoided in a tri-component-system by the use of suitable solvents and/or additives as described herein. An important precondition is the knowledge of the solubility of the osteoinductive active agent dependent on the nature of the solvent, i.e. aqueous and/or organic solvent, pH value, ionic strength and surfaces present.

The term “aqueous solution” specifies any solution comprising water. The slowing down of the pH increase caused by the contact of the coating solution with the calcium phosphates in the carrier reacting in an alkaline manner, in particular, plays an important role during the coating, preferably in method A.

The methods A and B of the present invention, distribute the active agent homogeneously across the inner surface of the carrier material and allow binding to the surface before a precipitation of the said protein takes place.

In method A this precipitation is pH-induced. It could be demonstrated that in this case the pH increase taking place during the coating of calcium phosphates is decelerated sufficiently by the use of a weak acid, such as acetic acid. Furthermore, the addition of organic compounds such as ethanol or sucrose proves to be additionally advantageous here. Furthermore, a low ionic strength is an important precondition for successful coating of the protein or peptide onto the calcium phosphate. Moreover, our tests show that the volume of the coating solutions (solution containing active agent and/or polymer), too, has a considerable effect on the quality of both coatings.

Finally, the methods A and B of the present invention allow the use of non toxic organic solvents (see below), such as dimethyl sulfoxide, anisol or glacial acid. These solvents are routinely used in the methods described in the art. Normally they damage the protein during contacting and/or especially during drying but such damage is surprisingly avoided by using the special drying technique of the present invention, because the active agent is adsorbed/or attached onto the inorganic solid carrier.

In a preferred embodiment of the method A of the invention said active agent coating buffer has a buffer concentration of preferably less than 100 mmol/l, more preferably less than 50 mmol/l and even more preferably less than 20 mmol/l to achieve a sufficient solubility of the active agent during the adsorption process and to avoid any modification of a ceramic carrier.

In another preferred embodiment of the method of the invention said buffer has a buffer concentration of 10 mmol/l to achieve a sufficient solubility of the active agent during the adsorption process and to avoid any modification of the monophasic calcium phosphate ceramic beta TCP. The pH of the solution shifts in a controlled manner during the coating and drying process from pH 3 to pH 7, more preferably from 3 to 6 and most preferably from 4 to 5.5. This pH shift causes a defined reduction of the solubility of the bone growth factor to result in a homogenous, defined attachment on the beta TCP.

In a preferred embodiment of the methods of the invention the solutions comprise non toxic organic solvents. The first aspect for method B is to find a common suitable organic solvent for both, the active agent and the polymer, without inducing modifications at the active agent. A second aspect is the ability of the solvent(s) in step (e) method A and/or step (a) of method B for the drying process, which is preferably a freeze-drying process. The preferred solvents are such as anisole, dimethylsulfoxide (DMSO) and glacial acetic acid. In a preferred embodiment these solvents in both method A and in method B are used in a volume to achieve a complete soaking of the polymer solution and to avoid any remaining solution.

It follows from the above that preferably, said buffer contains a weak acid. The term “weak acid” refers to organic or inorganic compounds containing at least one ionogenically bound hydrogen atom. Weak acids are well known in the art and are described in standard text books, such as Römpp, “dictionary of chemistry”. Preferably, said weak acids, which have low dissociation degrees and are described by pK values between 3 and 7, preferably between 4 and 6. Most preferably, said weak acid is acetic acid or succinic acid.

In another preferred embodiment of method A of the invention said buffer containing solution further comprises at least one saccharide in an aqueous solution, more preferably in an aqueous solution without any further solvent apart from water.

In a further preferred embodiment of the method of the invention said buffer containing solution comprises a polyol and/or alcohol. Suitable alcohols or polyols are well known in the art and are described in standard text books, such as Römpp, dictionary of chemistry. More preferably, said alcohol is ethanol and said polyol is mannitol.

In a more preferred embodiment the concentration of the polyol and or alcohol is between 0- and 10% (w/v).

The term “saccharides” encompasses mono-, di- and polysaccharides. The structure and composition of mono-, di-, and polysaccharides are well known in the art and are described in standard text books, such as Römpp, “dictionary of chemistry”.

More preferably, said saccharide is a disaccharide. Most preferably, said disaccharide is sucrose or trehalose.

Further means and methods for controlling homogeneous distribution, quantification and characterization of the active agent are described in the accompanied examples.

Surprisingly, active agents, in particular surprisingly proteins, when adsorbed on the surface of ceramic carriers are much more resistant against degradation caused by organic solvents than proteins freely dissolved or suspended in organic solutions or integrated in biphasic emulsion systems. Thus, this aspect of the invention opens a new possibility to produce polymer based drug delivery systems for proteins without denaturation and/or modification of polypeptides in singular or multiphase organic systems, preferably for active agents incompatible with organic solvents.

Furthermore the type of ceramic carrier preferably used in present invention opens the possibility to quantitatively remove organic solvents (see below).

Suitable for one of the two methods of the present invention as active agents are all proteins, polypeptides and small molecule drugs. Especially such active agents with low or no affinity for inorganic carrier matrices can be immobilized in the polymer—calcium phosphate composite material. Preferably, the binding of said active agent to the carrier is reversible.

Thereby, dissolution of said active agent is allowed once the material has been brought into a suitable in vivo surrounding, such as a bone cavity. More preferably, said dissolution of the immobilized compounds is a sustained release allowing diffusion of the active agent into the tissue, which surrounds the material. Thus, the material is suitable to serve as an in vivo source for e.g. osteoinductive proteins, peptides or small molecule drugs, which are slowly released and which can be thereby efficiently distributed into the surrounding tissues or have an effect in the immobilized form.

The term “drying” encompasses measures for removing liquids, such as excess buffer solution, or organic solvents, which are still present after coating of the carrier with the osteoinductive protein or polymer solution. Preferably, drying is achieved by convection at under inert gas atmosphere, by vacuum- or freeze-drying. It is important for the composite ceramic of the present invention that after drying the ceramic matrix is substantially free of organic solvent to allow for a softening of the polymeric component in a thermal treatment step, such as steps (g) of method A and step (e) of method B. Substantially free of organic solvent means a content preferably below ≦1% residual solvent, more preferably ≦0.05%, even more preferably ≦0.025% and most preferably ≦0.01%.

The term “buffer” which assists in keeping the active agent dissolved in aqueous solutions for a time sufficient to allow “homogenous coating” refers to a component allowing the active agent to be effectively dissolved in the solution and/or homogeneously coated in a carrier system tending to cause pH induced precipitation. This buffer is preferably capable of avoiding and or balancing the increase of pH caused by contacting the solution with the calcium phosphate carrier so that the protein does not immediately precipitate, e.g., due to a pH increase. Said buffer can be determined by the person skilled in the art considering the solubility of the osteoinductive protein (which depends on the pH and the ionic strength) and the influence of the carrier on said parameters after contacting the carrier with said buffer containing solution. In accordance with the present invention it has been found that a suitable buffer is needed for the homogeneous distribution of the active agent onto the surface of the carrier, e.g. calcium phosphate, said buffer comprising preferably a weak acid, an alcohol and a saccharide. The solvent for the dissolution of the preferably bioresorbable polymer in which the protein or peptide is not soluble described by the method A of the present invention comprises a suitable organic solvent for the homogeneous distribution of the polymer onto the surface of the protein or peptide coated carrier e.g. dimethylsulfoxide, anisol or glacial acid.

The term “thermal treatment” refers to a heating step which is applied after the solvent has been removed by drying to condense the polymeric phase by a definite collapse of the freeze dried structure and thus providing a dense and homogenous polymeric shell covering the ceramic surface of the granules. The purpose of this procedure is to modulate the release kinetics for the active substance and to achieve free flowing granules or to achieve the desired mechanical properties and manifestation of the composite material. By variation of the process conditions during the annealing step, the mechanical properties and the manifestation of the implant material can be fine tuned.

In accordance with the present invention, the composite carrier is based on a calcium phosphate and a polymer, preferably a biodegradable polymer. In such a composite carrier the calcium phosphate shows excellent local buffering capacity and the permeable composite structure avoids even local pH decrease when the polymer is degraded in vivo. Cytotoxic side effects due to degradation of the polymer are, hence, reduced or avoided. This is especially valid, since the ceramic carrier is chief ingredient of the ceramic carrier/polymer composite carrier material of the present invention, which preferably contains less than 60% of the polymer, most preferably less than 50% of PLGA even more preferably equal or less than 40% of PLGA.

In case of the calcium phosphate the ceramic carrier/polymer composite carrier material of the present invention preferably contains less than 100% of the calcium phosphate, more preferably 80%, most preferably less than 60% of calcium phosphate even more preferably equal or less than 50% calcium phosphate.

In case of an additionally filler material e.g. saccharides (Sucrose) salts (NaCl) or PEG to enhance the porosity of the ceramic carrier/polymer composite carrier material of the present invention preferably contains less than 60% of the filler material, most preferably less than 50% of the filler material even more preferably equal or less than 45% of filler material.

The temperature should be equal or higher than the glass transition temperature of the corresponding polymer system. For thermal sensitive active agents the glass transition temperature of the polymer can be decreased by the use of plasticizers, e.g. polyethylene glycol. The thermal treatment applies a temperature between the final drying temperature at ambient temperature, preferably ≧20° C., preferably ≧25° C., and most preferably ≧30° C. and the maximum temperature, limited by the active agent of ≦80° C., preferably ≦75° C., more preferably ≦65° C., and most preferably between 45° C. and 65° C. The time range for the thermal treatment in a preferred embodiment is as follows: Heating from 20° C. up to 60° C. in 30 minutes following by an isothermic period of about 50 minutes at this temperature. Afterwards the samples are cooled down to 20° C. for 1 hour. The integrity of the active agent was determined after extraction the polymeric shell as demonstrated in example 27.

The invention encompasses a pharmaceutical composition comprising the material of the invention or a material, which is obtainable by the method of the invention.

The product of the present invention can be formulated as a pharmaceutical composition or a medical material. The composition of said product may comprise additional compounds like stabilizers, buffer substances and other excipients. The amount of the product of the present invention applied to the patient will be determined by the attending physician and other clinical factors; preferably in accordance with any of the above described methods. As it is well known in the medical arts, the amount applied to a patient depends upon many factors, including the patient's size, body surface area, age, sex, time and route of administration, general health conditions, and other drugs being administered concurrently. Progress can be monitored by periodic assessment.

Thanks to the present invention, it is possible to treat various bone defects including large cavities in a new manner. In particular, large cavities could not or only under use of autogenous bone material be efficiently treated. However, due to the reliable and efficient osteoinductive and the osteoconductive properties of the material of the present invention or the material which can be obtained by the method of the invention, treatment of bone defects which requires extensive bone augmentation or repair has now become possible without a second surgery for gaining autologous bone material.

The invention also encompasses the use of the material of the invention or a material, which is obtainable by the method of the invention for the preparation of a pharmaceutical composition for bone augmentation.

The term “bone augmentation” refers to the induced formation of bone, which is indicated in order to treat bone defects, cavities in bones, or to treat diseases and disorders accompanied with loss of bone tissue or to prepare the subsequent setting of an implant. The diseases and disorders described in the following are well known in the art and are described in detail in standard medical text books such as Pschyrembel or Stedman.

Preferably, said bone augmentation follows traumatic, malignant or artificial defects.

Another embodiment of the present invention relates to the use of the material of the invention or the preparation of a pharmaceutical composition for treating bone defects.

More preferably, said bone defects are long bone defects or bone defects following apicoectomy, extirpation of cysts or tumors, tooth extraction, or surgical removal of retained teeth.

The invention also relates to the use of the material of the invention for filing of cavities and support guided tissue regeneration in periodontology.

Another embodiment of the present invention relates to the use of the material of the invention for the preparation of a pharmaceutical composition for sinus floor elevation, augmentation of the atrophied maxillary and mandibulary ridge and stabilization of immediate implants.

Also within the scope of the present invention is a method for treating one or more of the diseases referred to in accordance with the uses of the present invention, wherein said method comprises at least the step of administering the material of the invention in a pharmaceutically acceptable form to a subject. Preferably, said subject is a human.

Finally, the invention relates to a kit comprising the material of the invention.

The parts of the kit of the invention can be packaged individually in vials or other appropriate means depending on the respective ingredient or in combination in suitable containers or multicontainer units. Manufacture of the kit follows preferably standard procedures, which are known to the person skilled in the art.

### Contribution to the Field by the Present Invention

Polymer and carrier together form the ceramic/polymer composite carrier material of the present invention, which binds an osteoinductive active agent to result in the material of the present invention, in order to allow the sustained release of said active agent in vivo.

In accordance with the present invention, the composite material is based on a calcium phosphate and a polymer, preferably a biodegradable polymer. In such a composite carrier the calcium phosphate shows excellent local buffering capacity and the permeable composite structure avoids even local pH decrease when the polymer is degraded in vivo. Cytotoxic side effects due to degradation of the polymer are, hence, reduced or avoided.

This is especially valid, since the ceramic carrier is chief ingredient of the ceramic/polymer composite material of the present invention.

Thanks to the present invention, the polymer content within the composite material could be reduced by addition of a defined amount of the insoluble solid filler compared to conventional composite materials reducing the disadvantage of bulk degradation and pH alteration within the tissue resulting in improved biocompatibility of the material. Also, thanks to the present invention, dependent on the particle size of the water insoluble solid filler, the ratio of polymer to water insoluble solid filler (w/w), the water insoluble solid filler to polymer solution ratio (w/v) and the polymer concentration within the solution the method of the present invention enables to produce active agent encompassing composite materials such as free flowing granules.

Thanks to the present invention including a thermal treatment step into the process of manufacturing a compact surface coating of the composite material could be achieved (FIG. 2), which is less foamy and therefore less accessible to water diffusion into the material which enables a retarded degradation of the polymer and hence a retarded release of the active agent compared to conventional composites.

Furthermore, the ceramic/polymer composite carrier material of the present invention shows improved mechanical stability compared to conventional systems such as polymeric granules for retarded release. In one preferred embodiment, the resulting free flowing granules have mechanical properties, which are the same when compared with the untreated polymer free granules. These untreated granules represent the well established system to withstand tissue pressure in various therapies e.g. in orthopedic indications. In another preferred embodiment, the resulting composite material has mechanical properties, which exceeds the mechanical properties of known polymer based composites and purely polymer based scaffolds. In porous embodiments the composite matrix allows improved osteoconductive properties compared to prior art systems due to the porous system, in particular those free of interconnected pores.

The ceramic/polymer composite carrier material of the present invention is suitable to replace conventional encapsulating polymeric granules important for retarded release. Due to the homogeneous coating of the system (ceramic carrier plus polymer coating), the amount of polymer can be significantly reduced compared with other polymer based scaffolds, which reduced amount of polymer leads to a reduced risk of cytotoxicity. A further aspect of the invention is the increased mechanical stability of the composite material compared with other polymer based composites including totally polymer based scaffolds.

Thanks to the present invention, the process for manufacturing enables the production of homogenous coated active agent containing composite materials with advantages over state of the art composites: cost effective production due to processing of active non-degraded active agent without washing out and stressing the active agent while producing pores (e.g. salt leaching technique), titration of the release of the protein dependent on the polymer concentration used (FIG. 19 to 21), presence of active agent not only on the surface of the three-dimensional composite but also in the interior enabling release for a longer time compared to conventional composites. Without a combination of freeze drying and thermal treatment, the resulting composite would have areas of higher amounts of active agent in contrast to a homogenous coating and therefore unwanted high amounts of active agent which yields to unwanted biological responses e.g., a catabolic effect rather then an anabolic (FIG. 22).

The invention will now be described by reference to the following examples which are merely illustrative and which shall not limit the scope of the present invention. Some of the measures and results of the methods set forth in the examples can be obtained from the accompanying figures.

### EXAMPLES

### Example 1

### Manufacturing of β-TCP Granules with PLGA Shell (PLGA Content in the Final Material 4% w/w and 20% w/w)

500 mg β-TCP granules were coated by adding 425 μl of the corresponding PLGA (Resomer RG 502H), solution in DMSO, 21 mg (5% w/v) or 127.5 mg (30% w/v). The polymer coated granules are dried under the lyophilization conditions described in Table 2.

### Example 2

### Manufacturing Method of Composite Device Derived from β-TCP Granules

1.0 g β-TCP granules were submitted to the mould and 1.6 g polymer solution in acetic acid (15-30% w/v) was pipetted to the granules (until the meniscus locked up with the granules). During the preparation the mixture was evacuated and vented with air several times to ensure complete removal of entrapped air bubbles. This mixture was placed onto the pre-cooled plates of a freeze-dryer and dried under the lyophilization conditions described in Table 2.

### Example 3

### Manufacturing Method of Composite Device Derived from β-TCP Granules with Outer Dense Structure (Cage) to Support Mechanical Properties

1.0 g β-TCP granules were submitted in a polymer tube and 1.6 g polymer solution in acetic acid (15-30% w/v) was pipetted to the granules until the meniscus lock up with the granules. During the preparation the mixture was evacuated and vented with air several times to ensure complete removal of entrapped air bubbles. This mixture was placed onto the pre-cooled plates of a freeze-dryer and dried under the lyophilization conditions described in Table 2.

### Example 4

### Manufacturing Method of Composite Material Derived from β-TCP Powder

For samples with a PLGA/TCP ratio of 1.0:1.5 (0.7) 0.56 g β-TCP powder was submitted to a vessel and 1.26 g polymer solution in acetic acid (30% w/v). For samples with a PLGA/TCP ratio of 1.0:1.0 (1.0) 0.56 g β-TCP powder was submitted to a vessel and 1.87 g polymer solution in acetic acid (30% w/v). For samples with a PLGA/TCP ratio of 1.0:3.0 (0.3) 0.56 g β-TCP powder was submitted to a vessel and 0.63 g polymer solution in acetic acid (30% w/v).

The mixture was homogenized by mixing and evacuated and vented with air several times to ensure complete removal of entrapped air bubbles. The suspension was filled into a mould, placed onto the pre-cooled plates of a freeze-dryer and dried under the lyophilization conditions described in Table 2.

### Example 5

### Manufacturing Method of Composite Material Derived from TCP Powder with Outer Dense Structure (Cage) to Support Mechanical Properties

A suspension according to example 4 was filled in a polymer tube and evacuated and vented with air several times to ensure complete removal of entrapped air bubbles.

The sample was placed onto the pre-cooled plates of a freeze-dryer and dried under the lyophilization conditions described in Table 2.

### Example 6

### Manufacturing Method of Fiber Reinforced Composite Material Derived from β-TCP Powder

The fibers or fiber mesh (A. Glass fiber approximately 5 mm in length, loose fibers, B. PGA-fleece, rolled mesh, approx. 15×30×2.5 mm, C. Nylon, approx. 15×30×1 mm, rolled mash, D. Ethisorb, diameter 7×8 mm, cylinder mesh) were given into a mould and filled up with a suspension according to example 4.

The suspension/fiber mixture was evacuated and vented with air several times to ensure complete removal of entrapped air bubbles and placed onto the pre-cooled plates of a freeze dryer and dried under the lyophilization conditions described in Table 2.

### Example 7

### Analysing of the Composite Material by Scanning Electron Microscopy (SEM)

For analyzing the porosity and morphology of the composite material electron microscopy was used. The specimens were sputtered with gold. Thereby a vacuum of approximately 10⁻⁴ mbar was applied. The target structures for these analyses were the bottom and the core of the composite material derived from β-TCP powder.

### Example 8

### Determination of Porosity

The total porosity was determined by calculating the amount of the solvent in the material dispersion e.g., acetic acid before freeze-drying. After freeze-drying, the volume fraction of the solvent is equal to the total porosity of the material. The geometry of the composite material before and after thermal treatment was measured and the overall volume of the 3-dimensional scaffold was calculated. The difference between both volumes gave the relatively decrease of the porosity during the thermal treatment step.

### Example 9

### Mechanical Testing of Composite Material

For mechanical testing according ISO 604 each specimen was milled to a uniform height of 15 mm and 8 mm, respectively. The prepared specimen was loaded between two parallel plates on an electro servo hydraulic material testing system (TH 2730, Fa. Thümler, feed rate of 1 mm/sec) under displacement control. Young's modulus (E-modules) and compressive strength were calculated from the recorded compressive stress vs. compressive strain curves.

### Example 10

### Stability Testing of Pure rhGDF-5 after Drying from Various Organic Solvents

To analyze the effect of various organic solvents on the pure rhGDF-5 100 μg were dried under reduced pressure and 100 μl of the solvent were added. The samples were incubated for 1 hour dried again and subsequently heated at a temperature of 25° C. or 60° C. for another 1 hour. The rhGDF-5 were dissolved in extraction buffer and analyzed by RP-HPLC described in example 17 method A.

### Example 11

### Stability Testing of Pure PTH after Drying from Various Organic Solvents

To analyze the effect of various organic solvents on the pure PTH 20 μg were dried under reduced pressure and 100 μl of the solvent were added. The samples were incubated for 1 hour dried again and subsequently heated at a temperature of 25° C. or 60° C. for another 1 hour. The PTH were dissolved in extraction buffer and analyzed by RP-HPLC described in example 17 method B.

### Example 12

### Manufacturing of rhGDF-5 Coated β-TCP Granules

A. 500 mg β-TCP (0.5-1.0 mm granule size) are placed in a dry form in a 6R-glass. The stock solution of rhGDF-5 (4 mg/ml in 10 mM HCl) was diluted to 0.525 mg/ml rhGDF-5 in 10.0 mM acetic acid, 2.5 mM HCl, 10.0% sucrose. 475 μl of the rhGDF-5 solution obtained in that manner was pipetted on the beta-TCP and adsorbed. The damp granulate was then dried under the lyophilization conditions described in Table 1.

### Example 13

### Stability Testing of rhGDF-5 Coated β-TCP Granules in Various Organic Solvents

The amount of solvent induced protein degradation was determined by incubating 500 mg of rhGDF-5 coated granules according to example 12 with 425 μl of the solvent for 30 min. Afterwards the solvent were removed by evaporation under vacuum and analyzed by RP-HPLC described in example 16 and 17.

### Example 14

### Stability Testing of rhGDF-5 Coated β-TCP Granules after Drying from Various Organic Solvents and Annealing

The amount of solvent induced protein degradation during the annealing was determined by incubating 550 mg of rhGDF-5 coated granules according to example 12 with 425 μl of the solvent for 30 min. The solvent were removed by evaporation under vacuum and afterwards the vials were heated up at a temperature of 60° C. for 1 hour in an oven and analyzed by RP-HPLC described in example 16 and 17.

### Example 15

### Stability Testing of rhGDF-5 Coated β-TCP Granules after Drying from Various Organic Solvents and Annealing with Optimized Lyophilization Conditions

To measure the effect of the optimized manufacturing process on the solvent induced rhGDF-5 degradation 425 μl of the organic solvents were added to 550 mg rhGDF-5 coated granules according to example 12 incubated for 30 minutes and dried under the optimized lyophilization conditions described in Table 2. The rhGDF-5 degradation during manufacturing was quantified by RP-HPLC described in example 16 and 17.

### Example 16

### Extraction of the Immobilized rhGDF-5 Coated onto β-TCP Granules

200 mg rhGDF-5 coated granules according to example 12 were extracted in a 1 ml polypropylene reaction cup after resuspending in 1 ml extraction buffer (10 mM Tris, 100 mM EDTA, 8 M Urea, pH 6.7) under gentle agitation for 1 h at 4° C. After centrifugation (13 200 rpm g, 2 min) the supernatant was analyzed by RP-HPLC.

### Example 17

### Quantification and Determination of Chemical Modifications of the Protein and Peptide

Method A for Bone Growth Factor (rhGDF-5)

The amount of chemical modifications i.e. oxidation of bone growth factor in solutions containing extracted protein was determined by RP-HPLC. The sample was applied to a Vydak C8-18 column (2×250 mm) which has been equilibrated with 0.15% TFA, 20% acetonitrile. After washing of the column, the elution was performed with a mixture of 0.1% TFA, 20% acetonitrile, and a stepwise gradient of 20%-84% acetonitrile (flow: 0.3 ml/min). The elution was observed by measuring the absorption at 215 nm. The quantification was calculated by the ratio of the peak area of modified species to the total peak area.

### Method B for Peptide (PTH)

The amount of chemical modifications, i.e. oxidation of bone growth factor in solutions containing extracted protein was determined by RP-HPLC. The sample was applied to a Vydak C8-18 column (4.6×250 mm) which has been equilibrated with 0.15% TFA, 13.5% acetonitrile. After washing of the column, the elution was performed with a mixture of 0.1% TFA, 13.5% acetonitrile and a stepwise gradient of 13.5%-84% acetonitrile (flow: 0.5 ml/min). The elution was observed by measuring the absorption at 215 nm. The quantification was calculated by the ratio of the peak area of modified species to the total peak area.

### Example 18

### Manufacturing of Protein (rhGDF-5, Parathormone, PTH 1-34) Coated β-TCP Granules with PLGA Shell (PLGA Content in the Final Material 4% w/w and 20% w/w)

rhGDF-5 coated granules according to example 12 were coated by adding 425 μl of the corresponding PLGA (Resomer RG 502H), solution in DMSO, 21 mg (5% w/v) or 127.5 mg (30% w/v). The polymer coated granules were dried under the lyophilization conditions described in Table 2.

### Example 19

### Manufacturing of β-TCP Granules Coated with a Peptide (Parathormone, PTH 1-34) within the PLGA Shell (PLGA Content in the Final Material 20% w/w)

200 mg β-TCP granules were weight into a glass vial. The parathormone was diluted in DMSO to a final concentration of approx. 1.9 mg/ml. 44 μl of this parathormone solution was diluted with 891 μl PLGA solution 30% w/v in DMSO (RG 502H). The β-TCP was coated with 935 μl of the parathormone/PLGA solution and dried afterwards under the lyophilization conditions described in Table 2.

### Example 20

### Stability Testing of rhGDF-5 Coated β-TCP Granules with Various PLGA Shell Thickness after Drying and Annealing

(rhGDF-5) coated β-TCP granules with PLGA shell according to example 18 were taken. For example 100 mg of these granules were extracted with 1 ml of a saturated chloroform/lithium solution under gentle agitation for 1 hour at 4° C. to remove the PLGA shell. After centrifugation (13 000 rpm g, 3 min) the supernatant was separated and the residual granules was dried for 1 hour under reduced pressure.

Subsequently the granules were extracted and analyzed according to example 16 and 17

### Example 21

### Release Study of rhGDF-5 Coated β-TCP Granules with a PLGA Shell

150 mg of rhGDF-5 coated β-TCP granules with a PLGA shell according to example 18 were given into a 50 ml tube, 48 ml alpha-MEM-medium including 10% of FCS were added and incubated and gently rolled continuously at 4° C. for ≦7 days, (final concentration of the release-assay is ˜1.6 μg rhGDF-5/ml medium).

At pre-defined time aliquots of 100 μl were taken (the taken volume will not be replaced), centrifuged for 5 minutes at 13 000 rpm, the supernatant is frozen at −70° C. The quantification of rhGDF-5 in the selected aliquots was done by Elisa-assay according to Example 25.

### Example 22

### Manufacturing Method of rhGDF-5 Coated Composite Material Derived from β-TCP Powder

### Step 1:

500 mg β-TCP powder are placed in a dry form in a 6R-glass. The stock solution of rhGDF-5 (4 mg/ml in 10 mM HCl) is diluted to 0.525 mg/ml rhGDF-5 in 10.0 mM acetic acid, 2.5 mM HCl, 10.0% sucrose. 475 μl of the rhGDF-5 solution obtained in that manner are pipetted on the beta-TCP powder and adsorbed. The damp powder is then lyophilized.

### Step 2:

The rhGDF-5 coated β-TCP powder according to Step 1 and 1.1 g (1 ml) polymer solution in acetic acid (15-30%) was homogenized by mixing and evacuated and vented with air several times to ensure complete removal of entrapped air bubbles. The suspension was placed onto the pre-cooled plates of a freeze-dryer and dried under the lyophilization conditions described in Table 2.

### Example 23

### Manufacturing Method of rhGDF-5 Coated Composite Device Derived from β-TCP Granules

823 mg polymer solution in acetic acid (15-30%) was pipetted to 500 mg rhGDF-5 coated β-TCP granules according to example 12. This mixture was evacuated and vented with air several times to ensure complete removal of entrapped air bubbles. The sample was placed onto the pre-cooled plates of a freeze-dryer and dried under the lyophilization conditions described in Table 2.

### Example 24

### Release Study of rhGDF-5 Coated Composite Material Derived from Beta-TCP Powder

80-100 mg of rhGDF-5 coated composite material according to example 21 was given into a 50 ml tube, 48 ml alpha-MEM-medium including 10% of FCS were added and incubated and gently rolled continuously at 4° C. for ≦7 days, (final concentration of the release-assay is ˜0.8 μg rhGDF-5/ml medium).

At pre-defined time aliquots of 100 μl were taken (the taken volume will not be replaced), centrifuged for 5 minutes at 13 000 rpm, the supernatant is frozen at −70° C.

The quantification of rhGDF-5 in the selected aliquots will be done by Elisa-assay according to Example 25.

### Example 25

### Quantification of rhGDF-5 Release by ELISA

The rhGDF-5 release was quantified by means of ELISA. Initially antibody aMP-5 for rhGDF-5 was fixed on the surface of a microtiter plate. After having saturated free binding sites the plate was incubated with the samples containing rhGDF-5. Subsequently the bonded rhGDF-5 was incubated antibody aMP4, which was quantified by means of immune reaction with streptavidin POD.

### Example 26

### Quantification of rhGDF-5 in Solution by RP-HPLC

(rhGDF-5) coated β-TCP granules with PLGA shell according to example 18 were taken. For example 100 mg of these granules were extracted with 1 ml of a saturated chloroform/lithium solution under gentle agitation for 1 hour at 4° C. to remove the PLGA shell. After centrifugation (13 000 rpm, 3 min) the supernatant will be separated and the residual granules will be dried for 1 hour under reduced pressure. Subsequently the granules were extracted according to example 16.

The rhGDF-5 content was determined by reversed phase (RP-) HPLC-analysis. Aliquots of the sample were analysed using a Porous 10 R1 C4 column (self-packed). 0.045% trifluoroacetic acid in water (solvent A) and 0.025% trifluoroacetic acid in 84% acetonitrile (solvent B) were used as solvents at a flow rate of 0.4 ml/min. The elution profile was recorded by measuring the absorbance at 220 nm. The amounts of rhGDF-5 were calculated form the peak area at 220 nm using a standard curve.

### Example 27

### Detection of the Homogeneity of the Coating

The adsorbed protein is visualized by staining with Coomassie Brilliant Blue on beta-TCP granules as described in WO 03/043673. The distribution of the blue colour correlates with the distribution of the respective protein on the beta-TOP.

### Step 1:

(rhGDF-5) coated β-TCP granules with PLGA shell according to example 1 or 18 were taken. 100 mg of these granules were extracted with 2 times 1 ml of a saturated chloroform/lithium chloride solution under gentle agitation for 1 hour at 4° C. to remove the PLGA shell. After centrifugation (13 000 rpm g, 3 min) the supernatant was separated and the residual granules was dried for 1 hour under reduced pressure.

### Step 2:

3-4 coated granules were incubated with 200 μl staining solution (50% PBS, 40% methanol, 0.4% Coomassie Brilliant Blue R250) in a cavity of a 96-well plate and incubated for 30 min at room temperature. An uncoated carrier was treated in the same way as control. The surplus staining agent is removed by washing with 50% PBS, 40% methanol until the uncoated carrier used as control was completely destained. The stained carrier was dried at 40° C. and documented photographically.

### REFERENCES

Agrawal, C. M. et al. (1997). J. Biomed. Mater. Res. (Appl. Biomater.), 38: 105-114.
Bohner, M. (2001); Eur Spine J 10: S114-121.
Breitenbach J., (2002). Eur. J. Pharm. Biopharm. 54: 107-117.
Celeste A. J. et al. (1990). Proc. Natl. Acad. Sci. U.S.A. 87:9843-9847.
Chang, S. et al. (1994). J. Biol. Chem. 269: 28227-28234.
Cherng, A. et al. (1997); J Biomed Mater Res 35: 273-277.
Chow, L. C. (2000); Mater Res Symp Proc 599: 27-37.
Del Real, R. P. et al (2002); 23: 3673-3680.
Draenert, K. et al (2001). Trauma Berufskrankh. 3: 293-300.
Driessens, F. C. M. et al. (2002). Biomaterials 23: 4011-4017.
Dunn et al. U.S. Pat. No. 5,702,716
Durucan, C. et al. (2000). J. Biomed. Mater. Res. 51: 726-724.
EMEA, ICH Topic Q 3 C, Impurities: Residual Solvents
Friess, W. (1999). Eur J Pharm Biopharm 45: 113-36.
Gao, T. et al. (1996). Int. Orthopaedics 20: 321-325.
Gombotz, W. et al. (1996). In Formulation, characterization and stability of protein drugs, Plenum Press, New York, USA, pp 219-245.
Griffith, D. L. et al. (1996). Proc. Natl. Acad. Sci. U.S.A. 93: 878-883.
Guan, L.; Davies, J. E. (2004). J Biomed Mater Res A. 71: 480-7.
Herbert, P. et al. (1998). Pharm Res., 15: 357-361.
Hoetten, G. et al. (1994). Biochem. Biophys. Res. Commun. 204: 646-652.
Hollinger, J. O. et al. (1996). Biomaterials 17: 187-194.
Hotz, G. et al. (1994). Int. J. Oral Maxillofac. Surg. 23: 413-417.
Ignjatovic, N. L. et al. (1999). Biomaterials 20: 809-816.
Katagiri, T. et al. (1990). Biochem. Biophys. Res. Commun. 172: 295-299.
Kenley, R. A., et al. (1993). Pharm. Res. 10 (10) 1393-1401.
Khan, Y. M. et al. (2004). J Biomed Mater Res A. 15:728-37.
Kim, H. W. et al. (2004). Biomaterials. 2004 25:1279-87.
Li, S. M., et al. (1990). J. Mater. Sci. Mater. Med., 1, 123-130.
Lind, M. et al. (1996). J. Orthopaedic Res. 14, 343-350.
Lind, M. (1996). Acta. Orthop. Scand. 67: 407-17.
Middleton, J. C. et al. (2000). Biomaterials 21: 2335-2346.
Nishitoh, H. et al (1996). J. Biol. Chem. 271: 21345-21352.
Oezkayanak, E. et al. (1990). EMBO J. 9: 2085-2093.
Ramay, H. R. R.; Zhang, M. (2004). Biomaterials. 25:5171-80.
Ruhe, P. Q., et al. (2003); J Bone Joint surgery 85-A(3): 75-81.
Rueger, J. M. et al (1996). “Knochenersatzmittel”; Unfallchir. 99: 228-236.
Schiller, C. et al. (2003). Biomaterials 24:2037-2043.
Schmidmaier, G. et al. (2000). Chirurg 71: 1016-1022.
Schmitt, J. et al. (1999). J. Orthop. Res. 17: 269-278.
Seeherman, H. (2003). J. Bone. Joint. Surg. Am. 85-A Suppl. 3, 96-108
Shively M. L. et al. (1995). J. Controlled Rel. 33, 237-243.
Shore, E. M. et al. (1997). “Human bone morphogenetic protein-2 (BMP-2) genomic DNA sequence”.
Storm & Kingsley (1999). Development Biology, 209: 11-27.
Terheyden, H. et al. (1997). Mund Kiefer Gesichtschir. 1: 272-275.
Tormälä, P. et al. (1995). Mat. Clin. Appl. 639-651.
Tormälä, P. et al. (1992). Clin. Mater. 10: 29-34.
Vert, M. (1989). Angew. Makromol. Chem. 166/167: 155-168.
Wang, E. A. et al. (1990). Proc. Natl. Acad. Sci. U.S.A. 87: 9843-9847.
Wang, Z. G. et al. (2000). Polymer 41: 621-628.
Wintermantel, E. et al., Medizintechnik mit biokompatiblen Werkstoffen und Verfahren, page 7, 511, 3. Auflage, Springer 2002, ISBN 3-540-41261-1.
Wozney, J. M. et al. (1998). Clin. Orthop. 346: 26-37.
Wozney, J. M. et al. (1988). Science 242: 1528-1534.

1. Sterile pharmaceutical acceptable free flowing granules of a composite material or a sterile composite 3-dimensional scaffold comprising
a) a water insoluble solid filler,
b) a water insoluble polymer, and
c) an active agent homogenously dispersed within the polymer or homogeneously coated on the filler,
wherein the content of the intact active agent is equal to or more than 70%.

2. (canceled)

3. The sterile pharmaceutical acceptable free flowing granules of a composite material or composite 3-dimensional scaffold of claim 1, which is microporous,
wherein the polymer to carrier weight-ratio of the material is between 0.15 and 1 and the scaffold is obtained using a carrier comprising beta-tricalcium phosphate powder as educt.

4. The sterile pharmaceutical acceptable free flowing granules of a composite material or composite 3-dimensional scaffold of claim 1, which is macroporous,
wherein the polymer to carrier weight-ratio of the material is between 0.2 and 0.67 and the scaffold is obtained using a carrier consisting of beta-tricalcium phosphate granules as educt.

5. The sterile pharmaceutical acceptable free flowing granules of a composite material or composite 3-dimensional scaffold of claim 3,
wherein the polymer content is not more than 50 wt %, wherein the composite material has a compressive strength between 5 and 65 MPa and a Young's modulus of 15 to 30 MPa.

6. (canceled)

7. The sterile pharmaceutical acceptable free flowing granules or the composite 3-dimensional scaffold of claim 1, wherein the water insoluble solid carrier contains a calcium phosphate selected from beta tricalcium phosphate, alpha tricalcium phosphate, apatite and a calcium phosphate containing cement or a mixture of them.

8. (canceled)

9. (canceled)

10. The sterile pharmaceutical acceptable free flowing granules or the composite 3-dimensional scaffold of claim 1, wherein the active agent is an osteoinductive polypeptide.

11. A method for the production of a composite material comprising the steps of:
(a) providing an aqueous solution comprising an active agent and a buffer, which buffer keeps said active agent dissolved for a time sufficient to allow homogenous coating of a carrier, preferably a ceramic carrier when said carrier is contacted with said solution;
(b) contacting the solution of step (a) with a water insoluble solid carrier, preferably a ceramic carrier, more preferably a ceramic carrier containing calcium phosphate;
(c) allowing homogenous coating of the surface of said water insoluble solid carrier with said dissolved active agent;
(d) drying the coated water insoluble solid carrier obtained in step (c);
(e) providing a further solution comprising a dissolved water insoluble polymer or a mixture of water insoluble polymers, which polymer stays dissolved for a time sufficient to allow homogenous coating of the water insoluble solid carrier obtained in step (d) when said water insoluble solid carrier is contacted with said solution, wherein the water insoluble solid carrier and the active agent coated onto said water insoluble solid carrier is not soluble in said solution;
(f) freeze drying the polymer coated carrier obtained in step (e); and
(g) thermally treating said polymer coated carrier obtained in step (f), preferably under vacuum.

12. A method for the production of a composite material comprising the steps of:
(a) providing a solution comprising an active agent, and a water insoluble polymer or mixture of water insoluble polymers;
(b) contacting the solution of step (a) with a water insoluble solid carrier, preferably a ceramic carrier, more preferably a ceramic carrier containing calcium phosphate,
(c) allowing homogeneous coating of the surface of said carrier with said dissolved active agent and polymer;
(d) freeze drying the polymer coated carrier obtained in step (c); and
(e) thermally treating said coated carrier obtained in step (d), preferably under vacuum.

13. The method of claims 11 or 12, wherein the solution of claim 11 (e) and claim 12 (a) is a pharmaceutical acceptable organic solvent in which the polymer is soluble, which is compatible with the active agent, which is dryable under reduced pressure and removable by freeze drying.

14. (canceled)

15. The method of claims 11 or 12, wherein said water insoluble solid carrier contains a calcium phosphate selected from beta tricalcium phosphate, alpha tricalcium phosphate, apatite and a calcium phosphate containing cement.

16. (canceled)

17. (canceled)

18. The method of claims 11 or 12, wherein the freeze drying is performed under ambient temperature and thermal treating is performed above the glass transition temperature of the polymer system but below the denaturing temperature of the active agent.

19. The method of claims 11 or 12, wherein said biodegradable composite material is formed to exhibit a microporous solid three dimensional scaffold, preferably with the manifestation of a load bearing three-dimensional implant with mechanical properties preferably similar to trabecular bone, wherein the water insoluble carrier in step (b) of claims 11 or 12 comprises a powder form and the polymer content of the material is between 10 wt % and 50 wt %.

20. The method of claims 11 or 12, wherein said biodegradable composite material is formed to exhibit a macroporous solid three dimensional scaffold, preferably with the manifestation of a load bearing three-dimensional implant with mechanical properties preferably similar to trabecular bone, wherein the water insoluble carrier in step (b) of claims 11 or 12 consists of a granular form and the polymer content of the material is between 19 wt % and 45 wt %.

21. The method of claims 11 or 12, wherein said biodegradable composite material is formed to exhibit free flowing granules, wherein the water insoluble carrier in step (b) of claims 11 or 12 consists of a granular form and the polymer content of the material is between 0 wt % and 25 wt %.

22. The method of any one of claims 11 or 12, wherein said active agent is an osteoinductive polypeptide.

23. The method of any of claims 11 or 12, further comprising a step of hot pressing after the step of thermally treating.

24. The method of any of claims 11 or 12, further comprising a step of filling the polymer coated carrier obtained by step (e) of claim 11 or step (c) of claim 12 in an implant device and prosecuting the respective methods of claims 11 with step (f) and claim 12 with step (d) within the implant device.

25. The method of any of claims 11 or 12, further comprising a step of filling the polymer coated carrier obtained by step (d) of claim 11 into an implant device or performing step (b) of claim 12 with the water insoluble solid carrier, which has been filled into the implant device, and prosecuting the respective methods of claims 11 with step (e) and 12 with step (c) within the implant device.

26. A composite material, which is obtainable by the method of any one of claims 11 or 12.

27. A pharmaceutical composition comprising the composite material of claim 26.

28. A method for the preparation of a pharmaceutical composition for bone augmentation, for treating bone defects, degenerative and traumatic disc disease, bone dehiscence for filling cavities and/or support guided tissue regeneration in periodontology comprising preparing the sterile pharmaceutical acceptable free flowing granules or the composite 3-dimensional scaffold of claim 1.

29. (canceled)
